(19) United States
(12) Patent Application Publication
(10) Pub. No.: US 2004/0115671 A1
(43) Pub. Date: Jun. 17, 2004

(54) Gene expression profiling of endothelium in alzheimer's disease

(76) Inventors: Berislav V Zlokovic, Rochester, NY (US); Howard J Federoff, Rochester, NY (US)

Correspondence Address: NIXON & VANDERHYE, PC, 1100 N GLEBE ROAD, 8TH FLOOR, ARLINGTON, VA, 22201-4714, US

(21) Appl. No.: 10/451,917

(22) PCT Filed: Jan. 17, 2002
(86) PCT No.: PCT/US02/01069

(51) Int. Cl. C12Q 1/68; G06F 19/00; G01N 33/48; G01N 33/50

(52) U.S. Cl. 435/6; 702/20

(57) ABSTRACT

Changes in the gene expression profile of vascular endothelium are associated with or may be a cause for Alzheimer's disease. This observation can be used to diagnose the disease in symptomatic or asymptomatic individuals, to identify those at risk for disease or already affected thereby, to determine the stage of disease or the disease's progression, to intervene earlier in or alter the natural history of the disease, to provide targets for therapeutic or prophylactic treatments, to screen drugs or otherwise compare medical regimens, to determine the effectiveness of a drug or medical regimen, or any combination thereof. Gene expression may be profiled at the level of transcription (e.g., products like hnRNA, mRNA, and other RNA) and/or translation (e.g., products like nascent polypeptide, mature protein, and other processed or modified proteins).

### CROSS-REFERENCE TO RELATED APPLICATION

This application claims the benefit of provisional U.S. Appln. No. 60/262,064, filed Jan. 18, 2001.

### STATEMENT REGARDING FEDERAL SPONSORSHIP

The U.S. federal government has certain rights in this invention as provided for under NIH contract PO1 AG16233.

### FIELD OF THE INVENTION

The invention relates to the changes observed in vascular endothelium when gene expression is compared between patients affected by Alzheimer's disease and a control group without Alzheimer's disease. These observations can be used to diagnose the disease in symptomatic or asymptomatic individuals, to identify those at risk for the disease or those already affected thereby, to determine the stage of the disease or the disease's progression, to intervene earlier in or alter the disease's natural history, to provide targets for therapeutic or prophylactic treatments, to screen drugs or compare medical regimens, to determine the effectiveness of a drug or medical regimen in treating the disease, or any combination thereof. Gene expression may be profiled at the level of transcription (e.g., products like hnRNA, mRNA, and other RNA) and/or translation (e.g., products like nascent polypeptide, mature protein, and other processed or modified proteins).

### BACKGROUND OF THE INVENTION

Brain degenerative diseases associated with dysfunction of learning, memory, and/or cognition include cerebral senility, multiinfarct dementia, senile dementia of the Alzheimer type, age-associated memory impairment, and certain disorders associated with Parkinson's disease. Alzheimer's disease is the most common of the age-related neurodegenerative diseases: between about 10% and 20% of individuals over age 70 are affected, and about 50% of those over age 85 are affected. It is estimated that about 50% of nursing home residents in the U.S. are affected, and that annual costs associated with the care of patients with Alzheimer's disease in this country are in excess of $65 billion. As the U.S. population ages, the prevalence of Alzheimer's disease will increase dramatically from four million presently in the U.S. to more than 10 million by 2015. Study of the molecular basis of Alzheimer's disease complements behavioral studies. It can lead to a better understanding of pathogenesis and mechanisms of disease, as well as new modes of treatment.

Current dogma teaches that many different initiating events will ultimately cause synapses to fail to function properly and this leads inexorably to neuronal death. Several characteristic neuropathological findings are associated with Alzheimer's disease and the following can be considered indicia of the Alzheimer's phenotype: (a) intraneuronal deposits of neurofibrillary tangles (NFT), (b) parenchymal amyloid deposits—neuritic plaques, (c) cerebral amyloid angiopathy (CAA), and (d) synaptic loss. Popular current theories for the cause of Alzheimer's disease are the amyloid theory, the tau theory, and the inflammatory theory. Mutations in three genes encoding amyloid-β precursor protein (APP), presenilin-1, and presenilin-2 cause the rare, early-onset, autosomal dominant form of Alzheimer's disease. These mutations all affect APP metabolism such that more amyloid-β (Aβ) peptide is produced. In contrast, most cases of Alzheimer's disease have ages of onset above 65 years and exhibit no clear pattern of inheritance (i.e., late onset Alzheimer's disease or LOAD). The E4 allele of the apolipoprotein E (apoE) gene is the only known risk factor for LOAD. However, 50% of late-onset cases carry no apoE4 alleles, indicating that there must be additional risk factors. Recent studies have identified the focus for LOAD on chromosome 10 and linked it with increased levels of circulating Aβ₁₋₄₂. See refs. 1-10.

Deposition of Aβ in the CNS occurs during normal aging and is accelerated by Alzheimer's disease. Aβ is implicated in the neuropathology of Alzheimer's disease and related disorders. Recent studies suggest that the blood-brain barrier plays a role in determining the concentration of Aβ in the CNS. The blood-brain barrier has a dual role: (a) to control entry of plasma-derived Aβ and its binding/transport proteins into the CNS, and (b) to regulate levels of brain-derived Aβ via clearance mechanisms. See refs. 11-22.

Such genetic and biochemical approaches have neither taught nor suggested that Alzheimer's disease is associated with or may be caused by changes in the gene expression profile of brain endothelium (cf. St. George-Hyslop, Sci. Am. pp. 76-83, December 2000). Observations described below suggest that the Aβ peptide may not be the only toxin involved in pathogenesis of Alzheimer's disease. In particular, dysfunction of brain endothelium may cause and/or be the result of disease.

This observations can be used to improve our understanding of the pathogenesis of Alzheimer's disease and mechanisms of disease. Novel and inventive methods of diagnosis and treatment are suggested by these observations. Other advantages of the invention are discussed below or would be apparent from the disclosure herein.

References

1. Wisniewski et al. (1997) Neurobiol. Dis. 4:311-328.

2. Selkoe (1997) Science 275:630-631.

3. Selkoe (1998) Trends Cell Biol. 8:447-453.

4. Younkin (1998) J. Physiol. (Paris) 92:289-292.

5. Roses (1998) Amer. J. Med. Gen. 81:49-57.

6. Hardy et al. (1998) Nature Neurosci. 1:355-358.

7. Dickson (1997) J. Neuropathol. Exp. Neurol. 56:321-339.

8. Blacker et al. (1998) Nature Gen. 19:357-360.

9. Ertekin-Taner et al. (2000) Science 290:2303-2304.

10. Myers et al. (2000) Science 290:2304-2305.

11. Zlokovic (1997) Neurobiol. Dis. 4:23-26.

12. Zlokovic et al. (1993) Biochem. Biophys. Res. Commun. 197:1034-1040.

13. Maness et al. (1994) Life Sci. 55:1643-1650.

14. Poduslo et al. (1997) Neurobiol. Dis. 4:27-34.

15. Ghilardi et al. (1996) Neuroreport 7:2607-2611.

16. Mackic et al. (1998) J. Neurochem. 70:210-215.

17. Zlokovic et al. (1996) Proc. Natl. Acad. Sci. USA 93:4229-4236.

18. Martel et al. (1997) J. Neurochem. 69:1995-2004.

19. Shibata et al. (2000) J. Clin. Invest. 106:1489-1499.

20. Mackic et al. (1998) J. Clin. Invest. 102:734-743.

21. Zlokovic (1996) Life Sci. 59:1483-1497.

22. Ghersi-Egea et al. (1996) J. Neurochem. 67:880-883.

### SUMMARY OF THE INVENTION

In one embodiment of the invention, reagents are provided in kit form that can be used for performing the methods such as the following: diagnosis, identification of those at risk for disease or already affected, or determination of the stage of disease or its progression. In addition, the reagents may be used in methods related to the treatment of disease such as the following: evaluation whether or not it is desirable to intervene in the disease's natural history, alteration of the course of disease, early intervention to halt or slow progression, promotion of recovery or maintenance of function, provision of targets for beneficial therapy or prophylaxis, comparison of candidate drug, medical, or surgical regimens, or determination of the effectiveness of a drug, medical, or surgical regimen. The instructions for performing these methods, reference values and positive/negative controls, and relational databases containing patient information (e.g., genotype, medical history, symptoms, transcription or translation yields from gene expression, physiological or pathological findings) are other products considered to be aspects of the invention.

In other embodiments of the invention, the methods for diagnosis and treatment are provided. For screening of drugs and clinical trials, the respective drug and medical/surgical regimen selected are also considered to be embodiments of the invention. The amount and length of treatment administered to a cell, tissue, or individual in need of therapy or prophylaxis is effective in treating the affected cell, tissue, or individual. One or more properties/functions of affected endothelium or cells thereof, or the number/severity of symptoms of affected individuals, may be improved, reduced, normalized, ameliorated, or otherwise successfully treated. The invention may be used alone or in combination with other known methods. Instructions for performing these methods, reference values and positive/negative controls, and relational databases containing patient information are considered further aspects of the invention. The individual may be any animal or human. Mammals, especially humans and rodent or primate models of disease, may be treated; thus, both human and veterinary treatments are contemplated.

Further aspects of the invention will be apparent to a person skilled in the art from the following detailed description and claims, and generalizations thereto.

### BRIEF DESCRIPTION OF THE DRAWINGS

FIG. 1 shows a comparison of growth curves for primary cultures of microvascular brain endothelial cells (MBEC) from an Alzheimer's disease patient and a nondemented, normally-aged individual (FIG. 1A) and a comparison of population doubling times between the aforementioned cells (FIG. 1B). Circles (FIG. 1A) and a shaded bar (FIG. 1B) represent data from MBEC of the Alzheimer's disease patient; while squares (FIG. 1A) and a clear bar (FIG. 1B) represent data from MBEC of the nondemented, normally-aged individual.

MBEC were seeded at a density of 1×10³ cells per well in collagen I-coated 96-well plates. Cell counts were performed every day using triplicate sets of cultures that were trypsinized and then counted using a hemocytometer. Population doubling times were calculated as (T×ln 2)/ln(N₁/N₀), in which T is the length of time between the start and end points of log phase, N₀ is the cell number at the start of log phase, and N₁ is the cell number at the end of log phase. Statistical analysis shows that the difference in population doubling time between MBEC from the Alzheimer's disease patient and the age-matched control is significant with p=0.026.

FIG. 2 shows a possible model for relating the changes of gene expression profiles observed in Alzheimer's disease. The symbol in parentheses (+ or −) represents the general direction of the change (increase or decrease, respectively).

### DETAILED DESCRIPTION OF THE INVENTION

These studies are distinguished from previous neural and vascular theories for explaining the etiology of Alzheimer's disease because they focus on changes in gene expression of the endothelium and dysregulation of its physiology. Endothelial cells of brain microvessels, which are derived mainly from capillaries (about 90% to 95%) and a small percentage (about 5 to 10%) originating from smaller venules and arterioles (<20 μm diameter), have been studied. Here, a role for the endothelium is demonstrated which is different from the vascular theory of Alzheimer's disease that is mainly restricted to changes in Aβ transport through and clearance of Aβ from the brain, association of amyloid with blood vessels, and effects of Aβ on blood vessels.

Preparations of endothelial cells and endothelial cultures are provided from brain (e.g., microvasculature) or other organs (e.g., skin, blood vessels, bone marrow, blood containing endothelial precursors and stem cells) of individuals at risk for Alzheimer's disease, affected by the disease, or not. Tissue may be obtained as biopsy or autopsy material; cells of interest may be isolated therefrom and then cultured. Also provided are extracts of cells (e.g., cytoplasm, membrane); at least partially purified DNA, RNA, and protein therefrom; and methods for their isolation. These reagents can be used to establish detection limits for assays, absolute amounts of gene expression that are indicative of disease or not, ratios of gene expression that are indicative of disease or not, and the significance of differences in such values. These values for positive and/or negative controls can be measured at the time of assay, before an assay, after an assay, or any combination thereof. Values may be recorded on storage medium and manipulated with computer software; storage in a database allows retrospective or prospective study. For example, the database may be physically stored on a tangible media like note paper or plastic transparency, mechanical switch or electronic valve, iron core, semiconductor RAM or ROM, magnetic or optical disk, or paper or magnetic tape. The medium may be erased, refreshed (e.g., dynamic), or permanent (e.g., static); it may be fixed or transportable. Information may be displayed or projected on a screen (e.g., tangible media such as a cathode ray tube, light emitting diode, liquid crystal display). Genes that are increased, decreased, or not significantly changed in Alzheimer's disease are identified.

It is envisioned that the reliability of diagnostic methods may be improved by (1) decreasing the incidence of false positive and false negatives and (2) increasing the sensitivity of detection. For example, the number of different genes that have a measurable difference in expression (i.e., increased or decreased) may be at least about 5, 10, 15, 20, 25, 30, 35, 40, 45, 50, 60, 70, 80, 90, 100, 120, 140, 160, 180, 190, 200, 250, 300, 350, 400, 450, 500, 600, 700, 800, 900, 1000, 1200, 1400, 1600, 1800, 2000, 2200, 2400, 2600, 2800, 3000, 3200, 3400, 3600, 3800, 4000, 4200, 4400 or intermediate ranges thereof. The amount of change that is considered significant may be at least about 1.5-fold, 2-fold, 2.5-fold, 3-fold, 3.5-fold, 4-fold, 4.5-fold, 5-fold, 6-fold, 7-fold, 8-fold, 9-fold, 10-fold, 12-fold, 14-fold, 16-fold, 18-fold, 20-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, 100-fold, or intermediate ranges thereof as another example. The assay is quantitative in the sense that there is a direct and measurable relationship between the detected signal and the amount of gene expression (e.g., the number of transcripts or proteins), but the relationship does not necessarily need to be linear.

Polynucleotides representative of genes that are increased or decreased in Alzheimer's disease may be used to identify, isolate, or detect complementary polynucleotides by binding assays. Similarly, polypeptides representative of the gene products that are increased or decreased in Alzheimer's disease may be used to identify, isolate, or detect interacting proteins by binding assays. Optionally, bound complexes including interacting proteins may be identified, isolated, or detected indirectly though a specific binding molecule (e.g., antibody, natural or nonnatural peptide mimetic) for the gene product that is increased or decreased in Alzheimer's disease. Interacting proteins may also be associated with or cause Alzheimer's disease. Affinity chromatography of DNA-binding proteins, electrophoretic mobility shift assay (EMSA), one- or two-hybrid system, membrane protein cross-linking, and screening a phage display library may be used for identifying, isolating, or detecting interacting proteins. Candidate compounds useful for treating Alzheimer's disease may interact with a representative polynucleotide or polypeptide, and be screened for their ability to provide therapy or prophylaxis. These products may be used in assays (e.g., diagnosis) or for treatment; conveniently, they are packaged as assay kits or in pharmaceutical form.

Assaying Polynucleotides or Polypeptides

Binding of polynucleotides or polypeptides may take place in solution or on a substrate. The assay format may or may not require separation of bound from not bound. Detectable signals may be direct or indirect, attached to any part of a bound complex, measured competitively, amplified, or any combination thereof. A blocking or washing step may be interposed to improve sensitivity and/or specificity. Attachment of a polynucleotide or polypeptide, interacting protein, or specific binding molecule to a substrate before, after, or during binding results in capture of an unattached species. See U.S. Pat. Nos. 5,143,854 and 5,412,087.

Polynucleotide, polypeptide, or specific binding molecule may be attached to a substrate. The substrate may be solid or porous and it may be formed as a sheet, bead, fiber, tape, tube, or wire. The substrate may be made of cotton, silk, or wool; cellulose, nitrocellulose, nylon, or positively-charged nylon; natural, butyl, silicone, or styrenebutadiene rubber; agarose or polyacrylamide; crystalline silicon or polymerized organosiloxane; crystalline, amorphous, or impure silica (e.g., quartz) or silicate (e.g., glass); polyacrylonitrile, polycarbonate, polyethylene, polymethyl methacrylate, polymethylpentene, polypropylene, polystyrene, polysulfone, polytetrafluoroethylene, polyvinylidenefluoride, polyvinyl acetate, polyvinyl chloride, or polyvinyl pyrrolidone; or combinations thereof. Optically-transparent materials are preferred so that binding can be monitored and signal transmitted by light. For example, a bead suspended in solution and at the end of an optical fiber can be interrogated by a light signal (e.g., blue, red, or green) sent through the optical fiber when an analyte in solution (e.g., probe conjugated to a blue, red, or green label) binds to the bead, which is attached to the polynucleotide, polypeptide, or specific binding molecule. Such reagents would allow capture of a molecule in solution by specific binding, and then interaction of the molecule with and immobilization to the substrate. Monitoring gene expression is facilitated by using an ordered substrate array or coded library of multiple substrates.

Polynucleotide, polypeptide, or specific binding molecule may be synthesized in situ by solid-phase chemistry or photolithography to directly attach the nucleotides or amino acids to the substrate. Attachment of the polynucleotide, polypeptide, or specific binding molecule to the substrate may be through a reactive group as, for example, a carboxy, amino, or hydroxy radical; attachment may also be accomplished after contact printing, spotting with a pin, pipetting with a pen, or spraying with a nozzle directly onto a substrate. Alternatively, the polynucleotide, polypeptide, or specific binding molecule may be reversibly attached to the substrate by interaction of a specific binding pair (e.g., antibody-digoxygenin/hapten/peptide epitope, biotin-avidin/streptavidin, glutathione S transferase or GST-glutathione, lectin-sugar, maltose binding protein-maltose, polyhistidine-nickel, protein A/G-immunoglobulin); cross-linking may be used if irreversible attachment is desired.

By synthesizing the polynucleotide, polypeptide, or specific binding molecule in situ or otherwise attaching it to a substrate at a predetermined, discrete position or to a coded substrate, an interacting polynucleotide, polypeptide, or specific binding molecule can be identified without determining its sequence. For example, a polynucleotide, polypeptide, or specific binding molecule of known sequence can be determined by its position (e.g., rectilinear or polar coordinates) or decoding its signal (e.g., combinatorial tag, electromagnetic radiation) on the substrate. A nucleotide or amino acid sequence will be correlated with each position on or decoded signal of the substrate. A substrate may have a pattern of different polynucleotides, polypeptides, and/or specific binding molecules (e.g., at least 5, 10, 20, 30, 40, 50, 60, 80, 100, 150, 200, 250, 300, 350, 400, 450, 500, 1000, 2000, 3000, 4000, 5000, 7500, 10,000, 50,000, 100,000 or 1,000,000 distinguishable positions) at low or high density (e.g., at least 1,000, 10,000, 100,000 or 1,000,000 distinguishable positions per cm²). The number of sequences that can be differentiated by the signal is only limited by factors such as the number and complexity of combinations; interference between a property of electromagnetic radiation like wavelength, frequency, energy, polarization; etc.

Multiplex analysis may be used to monitor expression of different genes at the same time in parallel. Such multiplex analysis may be performed using different polynucleotides, polypeptides, or specific binding molecules arranged in high density on a substrate. Simultaneous solution methods such as multiprobe ribonuclease protection assay or multiprimer pair amplification associate each transcript with a different length of detected product which is resolved by separation on the basis of molecular weight.

Changes in gene expression may be manifested in the cell by affecting transcriptional initiation, transcript stability, translation of transcript into protein product, protein stability, or a combination thereof. The gene, transcript, or polypeptide can be assayed by techniques such as in vitro transcription, in vitro translation, Northern hybridization, nucleic acid hybridization, reverse transcription-polymerase chain reaction (RT-PCR), run-on transcription, Southern hybridization, cell surface protein labeling, metabolic protein labeling, antibody binding, immunoprecipitation (IP), enzyme linked immunosorbent assay (ELISA), electrophoretic mobility shift assay (EMSA), radioimmunoassay (RIA), fluorescent or histochemical staining, microscopy and digital image analysis, and fluorescence activated cell analysis or sorting (FACS).

A reporter or selectable marker gene whose protein product is easily assayed may be used for convenient detection. Reporter genes include, for example, alkaline phosphatase, β-galactosidase (LacZ), chloramphenicol acetyltransferase (CAT), β-glucoronidase (GUS), bacterial/insect/marine invertebrate luciferases (LUC), green and red fluorescent proteins (GFP and RFP, respectively), horseradish peroxidase (HRP), β-lactamase, and derivatives thereof (e.g., blue EBFP, cyan ECFP, yellow-green EYFP, destabilized GFP variants, stabilized GFP variants, or fusion variants sold as LIVING COLORS fluorescent proteins by Clontech). Reporter genes would use cognate substrates that are preferably assayed by a chromogen, fluorescent, or luminescent signal. Alternatively, assay product may be tagged with a heterologous epitope (e.g., FLAG, MYC, SV40 T antigen, glutathione transferase, hexahistidine, maltose binding protein) for which cognate antibodies or affinity resins are available.

A polynucleotide may be ligated to a linker oligonucleotide or conjugated to one member of a specific binding pair (e.g., antibody-digoxygenin/hapten/peptide epitope, biotin-avidin/streptavidin, glutathione S transferase or GST-glutathione, lectin-sugar, maltose binding protein-maltose, polyhistidine-nickel, protein A/G-immunoglobulin). The polynucleotide may be conjugated by ligation of a nucleotide sequence encoding the binding member. A polypeptide may be joined to one member of the specific binding pair by producing the fusion encoded such a ligated or conjugated polynucleotide or, alternatively, by direct chemical linkage to a reactive moiety on the binding member by chemical cross-linking. Such polynucleotides and polypeptides may be used as an affinity reagent to identify, to isolate, and to detect interactions that involve specific binding of a transcript or protein product of the expression vector. Before or after affinity binding of the transcript or protein product, the member attached to the polynucleotide or polypeptide may be bound to its cognate binding member. This can produce a complex in solution or immobilized to a support. A protease recognition site (e.g., for enterokinase, Factor Xa, ICE, secretases, thrombin) may be included between adjoining domains to permit site specific proteolysis that separates those domains and/or inactivates protein activity.

Construction of Expression Vector

An expression vector is a recombinant polynucleotide that is in chemical form either a deoxyribonucleic acid (DNA) and/or a ribonucleic acid (RNA). The physical form of the expression vector may also vary in strandedness (e.g., single-stranded or double-stranded) and topology (e.g., linear or circular). The expression vector is preferably a double-stranded deoxyribonucleic acid (dsDNA) or is converted into a dsDNA after introduction into a cell (e.g., insertion of a retrovirus into a host genome as a provirus). The expression vector may include one or more regions from a mammalian gene expressed in the microvasculature, especially endothelial cells (e.g., ICAM-2, tie), or a virus (e.g., adenovirus, adeno-associated virus, cytomegalovirus, fowlpox virus, herpes simplex virus, lentivirus, Moloney leukemia virus, mouse mammary tumor virus, Rous sarcoma virus, SV40 virus, vaccinia virus), as well as regions suitable for genetic manipulation (e.g., selectable marker, linker with multiple recognition sites for restriction endonucleases, promoter for in vitro transcription, primer annealing sites for in vitro replication). The expression vector may be associated with proteins and other nucleic acids in a carrier (e.g., packaged in a viral particle) or condensed with chemicals (e.g., cationic polymers) to target entry into a cell or tissue.

The expression vector further comprises a regulatory region for gene expression (e.g., promoter, enhancer, silencer, splice donor and acceptor sites, polyadenylation signal, cellular localization sequence). Transcription can be regulated by tetracyline or dimerized macrolides. The expression vector may be further comprised of one or more splice donor and acceptor sites within an expressed region; Kozak consensus sequence upstream of an expressed region for initiation of translation; and downstream of an expressed region, multiple stop codons in the three forward reading frames to ensure termination of translation, one or more mRNA degradation signals, a termination of transcription signal, a polyadenylation signal, and a 3′ cleavage signal. For expressed regions that do not contain an intron (e.g., a coding region from a cDNA), a pair of splice donor and acceptor sites may or may not be preferred. It would be useful, however, to include mRNA degradation signal(s) if it is desired to express one or more of the downstream regions only under the inducing condition. An origin of replication may also be included that allows replication of the expression vector integrated in the host genome or as an autonomously replicating episome. Centromere and telomere sequences can also be included for the purposes of chromosomal segregation and protecting chromosomal ends from shortening, respectively. Random or targeted integration into the host genome is more likely to ensure maintenance of the expression vector but episomes could be maintained by selective pressure or, alternatively, may be preferred for those applications in which the expression vector is present only transiently.

An expressed region may be derived from any gene of interest, and be provided in either orientation with respect to the promoter; the expressed region in the antisense orientation will be useful for making cRNA and antisense polynucleotide. The gene may be derived from the host cell or organism, from the same species thereof, or designed de novo; but it is preferably of archael, bacterial, fungal, plant, or animal origin. The gene may have a physiological function of one or more nonexclusive classes: adhesion proteins; cytokines, hormones, and other regulators of cell growth, mitosis, meiosis, apoptosis, differentiation, or development; soluble or membrane receptors for such factors; adhesion molecules; cell-surface receptors and ligands thereof; cytoskeletal and extracellular matrix proteins; cluster differentiation (CD) antigens, antibody and T-cell antigen receptor chains, histocompatibility antigens, and other factors mediating specific recognition in immunity; chemokines, receptors thereof, and other factors involved in inflammation; enzymes producing lipid mediators of inflammation and regulators thereof; clotting and complement factors; ion channels and pumps; transporters and binding proteins; neurotransmitters, neurotrophic factors, and receptors thereof; cell cycle regulators, oncogenes, and tumor suppressors; other transducers or components of signaling pathways; proteases and inhibitors thereof; catabolic or metabolic enzymes, and regulators thereof. Some genes produce alternative transcripts, encode subunits that are assembled as homopolymers or heteropolymers, or produce propeptides that are activated by protease cleavage. The expressed region may encode a translational fusion; open reading frames of the regions encoding a polypeptide and at least one heterologous domain may be ligated in register. If a reporter or selectable marker is used as the heterologous domain, then expression of the fusion protein may be readily assayed or localized. The heterologous domain may be an affinity or epitope tag.

Screening of Candidate Compounds

Another aspect of the invention are chemical or genetic compounds, derivatives thereof, and compositions including same that are effective in treatment of Alzheimer's disease and individuals at risk thereof. The amount that is administered to an individual in need of therapy or prophylaxis, its formulation, and the timing and route of delivery is effective to reduce the number or severity of symptoms, to slow or limit progression of symptoms, to inhibit expression of one or more genes that are transcribed at a higher level in Alzheimer's disease, to activate expression of one or more genes that are transcribed at a lower level in Alzheimer's disease, or any combination thereof. Determination of such amounts, formulations, and timing and route of drug delivery is within the skill of persons conducting in vitro assays, in vivo studies of animal models, and human clinical trials.

A screening method may comprise administering a candidate compound to an organism or incubating a candidate compound with a cell, and then determining whether or not gene expression is modulated. Such modulation may be an increase or decrease in activity that partially or fully compensates for a change that is associated with or may cause Alzheimer's disease. Gene expression may be increased at the level of rate of transcriptional initiation, rate of transcriptional elongation, stability of transcript, translation of transcript, rate of translational initiation, rate of translational elongation, stability of protein, rate of protein folding, proportion of protein in active conformation, functional efficiency of protein (e.g., activation or repression of transcription), or combinations thereof. See, for example, U.S. Pat. Nos. 5,071,773 and 5,262,300. High-throughput screening assays are possible (e.g., by using parallel processing and/or robotics).

The screening method may comprise incubating a candidate compound with a cell containing a reporter construct, the reporter construct comprising transcription regulatory region covalently linked in a cis configuration to a downstream gene encoding an assayable product; and measuring production of the assayable product. A candidate compound which increases production of the assayable product would be identified as an agent which activates gene expression while a candidate compound which decreases production of the assayable product would be identified as an agent which inhibits gene expression. See, for example, U.S. Pat. Nos. 5,849,493 and 5,863,733.

The screening method may comprise measuring in vitro transcription from a reporter construct in the presence or absence of a candidate compound (the reporter construct comprising a transcription regulatory region) and then determining whether transcription is altered by the presence of the candidate compound. In vitro transcription may be assayed using a cell-free extract, partially purified fractions of the cell, purified transcription factors or RNA polymerase, or combinations thereof. See, for example, U.S. Pat. Nos. 5,453,362; 5,534,410; 5,563,036; 5,637,686; 5,708,158; and 5,710,025.

Techniques for measuring transcriptional or translational activity in vivo are known in the art. For example, a nuclear run-on assay may be employed to measure transcription of a reporter gene. Translation of the reporter gene may be measured by determining the activity of the translation product. The activity of a reporter gene can be measured by determining one or more of transcription of polynucleotide product (e.g., RT-PCR of GFP transcripts), translation of polypeptide product (e.g., immunoassay of GFP protein), and enzymatic activity of the reporter protein per se (e.g., fluorescence of GFP or energy transfer thereof).

Genetic Compounds for Treatment

Gene activation may be achieved by inducing an expression vector containing a downstream region related to a gene that is down regulated (e.g., the full-length coding region or functional portions of the gene; hypermorphic mutants, homologs, orthologs, or paralogs thereof) or unrelated to the gene that acts to relieve suppression of gene activation (e.g., at least partially inhibiting expression of a negative regulator of the gene). Overexpression of transcription or translation, as well as overexpressing protein function, is a more direct approach to gene activation. Alternatively, the downstream expressed region may direct homologous recombination into a locus in the genome and thereby replace an endogenous transcriptional regulatory region of the gene with an expression cassette.

An expression vector may be introduced into a host mammalian cell or tissue, or nonhuman mammal by a transfection or transgenesis technique using, for example, one or more chemicals (e.g., calcium phosphate, DEAE-dextran, lipids, polymers), biolistics, electroporation, naked DNA technology, microinjection, or viral infection. Osmotic shock or surgical procedures may also be used for transfer across the blood-brain barrier. The introduced expression vector may integrate into the host genome of the mammalian cell or nonhuman mammal, or be maintained as an episome. Many neutral and charged lipids, sterols, and other phospholipids to make lipid carriers are known. For example, neutral lipids are dioleoyl phosphatidylcholine (DOPC) and dioleoyl phosphatidyl ethanolamine (DOPE); an anionic lipid is dioleoyl phosphatidyl serine (DOPS); cationic lipids are dioleoyl trimethyl ammonium propane (DOTAP), dioctadecyldiamidoglycyl spermine (DOGS), dioleoyl trimethyl ammonium (DOTMA), and 1,3-dioleoyloxy-2-(6-carboxyspermyl)-propylamide tetraacetate (DOSPER). Dipalmitoyl phosphatidylcholine (DPPC) can be incorporated to improve the efficacy and/or stability of delivery. FUGENE 6, LIPOFECTAMINE, LIPOFECTIN, DMRIE-C, TRANSFECTAM, CELLFECTIN, PFX-1, PFX-2, PFX-3, PFX4, PFX-5, PFX-6, PFX-7, PFX-8, TRANSFAST, TFX-10, TFX-20, TFX-50, and LIPOTAXI lipids are proprietary formulations. The polymer may be cationic dendrimers, polyamides, polyamidoamines, polyethylene or polypropylene glycols (PEG), polyethylenimines (PEI), polylysines, or combinations thereof; alternatively, polymeric materials can be formed into nanoparticles or microparticles. In naked DNA technology, the expression vector (usually as a plasmid) is delivered to a cell or tissue, where it may or may not become integrated into the host genome, without using chemical transfecting agents (e.g., lipids, polymers) to condense the expression vector prior to its introduction into the cell or tissue.

A mammalian cell may be transfected; also provided are transgenic nonhuman mammals. In the previously discussed alternative, a homologous region from a gene can be used to direct integration to a particular genetic locus in the host genome and thereby regulate expression of the gene at that locus or ectopic copies of the gene may be inserted. Polypeptide may be produced in vitro by culturing transfected cells, in vivo by transgenesis, or ex vivo by introducing an expression vector into allogeneic, autologous, histocompatible, or xenogeneic cells and then transplanting the transfected cells into a host organism. Special harvesting and culturing protocols will be needed for transfection and subsequent transplantation of host stem cells into a host mammal. Immunosuppression of the host mammal post-transplant or encapsulation of the host cells may be necessary to prevent rejection.

The expression vector may be used to replace function of a gene that is down regulated or totally defective, supplement function of a partially defective gene, or compete with activity of the gene. Thus, the cognate gene activity of the host may be neomorphic, hypomorphic, hypermorphic, or normal. Replacement or supplementation of function can be accomplished by the methods discussed above, and transfected mammalian cells or transgenic nonhuman mammals may be selected for high or low expression (e.g., assessing amount of transcribed or translated produce, or physiological function of either product) of the downstream region. But competition between the expressed downstream region and a neomorphic, hypermorphic, or normal gene may be more difficult to achieve unless the encoded polypeptides are multiple subunits that form into a polymeric protein complex. Alternatively, a negative regulator or a single-chain antibody that inhibits function intracellularly may be encoded by the downstream region of the expression vector. Therefore, at least partial inhibition of genes that are up regulated in MBEC of Alzheimer's disease may use antisense, ribozyme, RNAi, or triple helix technology in which the expression vector contains a downstream region corresponding to the unmodified antisense molecule, ribozyme, siRNA duplex, or triple helix molecule, respectively.

Antisense polynucleotides were initially believed to directly block translation by hybridizing to mRNA transcripts, but may involve degradation of such transcripts of a gene. The antisense molecule may be recombinantly made using at least one functional portion of a gene in the antisense orientation as a downstream expressed region in an expression vector. Chemically modified bases or linkages may be used to stabilize the antisense polynucleotide by reducing degradation or increasing half-life in the body (e.g., methyl phosphonates, phosphorothioate, peptide nucleic acids). The sequence of the antisense molecule may be complementary to the translation initiation site (e.g., between −10 and +10 of the target's nucleotide sequence).

Ribozymes catalyze specific cleavage of an RNA transcript or genome. The mechanism of action involves sequence-specific hybridization to complementary cellular or viral RNA, followed by endonucleolytic cleavage. Inhibition may or may not be dependent on ribonuclease H activity. The ribozyme includes one or more sequences complementary to the target RNA as well as catalytic sequences responsible for RNA cleavage (e.g., hammerhead, hairpin, axehead motifs). For example, potential ribozyme cleavage sites within a subject RNA are initially identified by scanning the subject RNA for ribozyme cleavage sites which include the following trinucleotide sequences: GUA, GUU and GUC. Once identified, an oligonucleotide of between about 15 and about 20 ribonucleotides corresponding to the region of the subject RNA containing the cleavage site can be evaluated for predicted structural features, such as secondary structure, that can render candidate oligonucleotide sequences unsuitable. The suitability of candidate sequences can then be evaluated by their ability to hybridize and cleave target RNA.

siRNA refers to double-stranded RNA of at least 20-25 basepairs which mediates RNA interference (RNAi). Duplex siRNA corresponding to a target RNA may be formed by separate transcription of the strands, coupled transcription from a pair of promoters with opposing polarities, or annealing of a single RNA strand having an at least partially self-complementary sequence. Alternatively, duplexed oligoribonucleotides of at least 21-23 basepairs may be chemically synthesized (e.g., a duplex of 21 ribonucleotides with 3′ overhangs of two ribonucleotides) with some substitutions by modified bases being tolerated. Mismatches in the center of the siRNA sequence, however, abolishes interference. The region targeted by RNA interference should be transcribed, preferably as a coding region of the gene. Interference appears to be dependent on cellular factors (e.g., ribonuclease III) that cleave target RNA at sites 21 to 23 bases apart; the position of the cleavage site appears to be defined by the 5′ end of the guide siRNA rather than its 3′ end. Priming by a small amount of siRNA may trigger interference after amplification by an RNA-dependent RNA polymerase.

Molecules used in triplex helix formation for inhibiting expression of a gene that is up regulated should be single-stranded and composed of deoxyribonucleotides. The base composition of these oligonucleotides must be designed to promote triple helix formation by Hoogsteen base pairing rules, which generally require sizeable stretches of either purines or pyrimidines to be present on one strand of the duplex. Nucleotide sequences can be pyrimidine-based and result in TAT and CGC triplets across the three associated strands. The pyrimidine-rich molecules provide base complementarity to a purine-rich region of a single strand of the duplex in a parallel orientation to that strand. In addition, triple helix forming molecules can be chosen that are purine-rich (e.g., containing a stretch of guanines). These molecules may form a triple helix with a DNA duplex that is rich in GC pairs, in which the majority of the purines are located on a single strand of the targeted duplex, resulting in GGC triplets across the three strands in the triplex.

Antibody specific for a gene product increased in Alzheimer's disease can be used for inhibition or detection. Polyclonal or monoclonal antibodies may be prepared by immunizing animals (e.g., chicken, hamster, mouse, rat, rabbit, goat, horse) with antigen, and optionally affinity purified against the same or a related antigen. Antibody fragments may be prepared by proteolytic cleavage or genetic engineering; humanized antibody and single-chain antibody may be prepared by transplanting sequences from the antigen binding domains of antibodies to framework molecules. In general, other specific binding molecules may be prepared by screening a combinatorial library for a member which specifically binds antigen (e.g., phage display library). Antigen may be a full-length protein encoded by the gene or fragment(s) thereof. See, for example, U.S. Pat. Nos. 5,403,484; 5,723,286; 5,733,743; 5,747,334; and 5,871,974.

Formulation of Compositions

Compounds of the invention or derivatives thereof may be used as a medicament or used to formulate a pharmaceutical composition with one or more of the utilities disclosed herein. They may be administered in vitro to cells in culture, in vivo to cells in the body, or ex vivo to cells outside of an individual which may then be returned to the body of the same individual or another. Such cells may be disaggregated or provided as solid tissue.

Compounds or derivatives thereof may be used to produce a medicament or other pharmaceutical compositions. Use of compositions which further comprise a pharmaceutically acceptable carrier and compositions which further comprise components useful for delivering the composition to an individual are known in the art. Addition of such carriers and other components to the composition of the invention is well within the level of skill in this art.

Pharmaceutical compositions may be administered as a formulation adapted for passage through the blood-brain barrier or direct contact with the endothelium. Alternatively, pharmaceutical compositions may be added to the culture medium. In addition to the active compound, such compositions may contain pharmaceutically-acceptable carriers and other ingredients known to facilitate administration and/or enhance uptake (e.g., saline, dimethyl sulfoxide, lipid, polymer, affinity-based cell specific-targeting systems). The composition may be incorporated in a gel, sponge, or other permeable matrix (e.g., formed as pellets or a disk) and placed in proximity to the endothelium for sustained, local release. The composition may be administered in a single dose or in multiple doses which are administered at different times.

Pharmaceutical compositions may be administered by any known route. By way of example, the composition may be administered by a mucosal, pulmonary, topical, or other localized or systemic route (e.g., enteral and parenteral). The term “parenteral” includes subcutaneous, intradermal, intramuscular, intravenous, intra-arterial, intrathecal, and other injection or infusion techniques, without limitation.

Suitable choices in amounts and timing of doses, formulation, and routes of administration can be made with the goals of achieving a favorable response in the individual with Alzheimer's disease or at risk thereof (i.e., efficacy), and avoiding undue toxicity or other harm thereto (i.e., safety). Therefore, “effective” refers to such choices that involve routine manipulation of conditions to achieve a desired effect.

A bolus of the formulation administered to an individual over a short time once a day is a convenient dosing schedule. Alternatively, the effective daily dose may be divided into multiple doses for purposes of administration, for example, two to twelve doses per day. Dosage levels of active ingredients in a pharmaceutical composition can also be varied so as to achieve a transient or sustained concentration of the compound or derivative thereof in an individual, especially in and around vascular endothelium of the brain, and to result in the desired therapeutic response or protection. But it is also within the skill of the art to start doses at levels lower than required to achieve the desired therapeutic effect and to gradually increase the dosage until the desired effect is achieved.

The amount of compound administered is dependent upon factors known to a person skilled in the art such as bioactivity and bioavailability of the compound (e.g., half-life in the body, stability, and metabolism); chemical properties of the compound (e.g., molecular weight, hydrophobicity, and solubility); route and scheduling of administration; and the like. For systemic administration, passage of the compound or its metabolite through the bloodbrain barrier is important. It will also be understood that the specific dose level to be achieved for any particular individual may depend on a variety of factors, including age, gender, health, medical history, weight, combination with one or more other drugs, and severity of disease.

The term “treatment” of Alzheimer's disease refers to, inter alia, reducing or alleviating one or more symptoms in an individual, preventing one or more symptoms from worsening or progressing, promoting recovery or improving prognosis, and/or preventing disease in an individual who is free therefrom as well as slowing or reducing progression of existing disease. For a given individual, improvement in a symptom, its worsening, regression, or progression may be determined by an objective or subjective measure. Efficacy of treatment may be measured as an improvement in morbidity or mortality (e.g., lengthening of survival curve for a selected population). Prophylactic methods (e.g., preventing or reducing the incidence of relapse) are also considered treatment. Treatment may also involve combination with other existing modes of treat-ment (e.g., ARICEPT or donepezil, COGNEX or tacrine, EXELON or rivastigmine, REMINYL or galantamine, anti-amyloid vaccine, mental exercise or stimulation). Thus, combination treatment with one or more other drugs and one or more other medical procedures may be practiced.

Similarly, diagnosis according to the invention may be practiced with other diagnostic procedures. For example, endothelium of the blood, brain, or spinal cord (e.g., blood or leptomeningeal vessels) may be assayed for a change in gene expression profiles. In addition, a noninvasive diagnostic procedure (e.g., CAT, MRI or PET) may be used in combination to improve the accuracy and/or sensitivity of diagnosis. Early and reliable diagnosis is especially useful to for treatments that are only effective for mild to moderate Alzheimer's disease or only delay its progression.

The amount which is administered to an individual is preferably an amount that does not induce toxic effects which outweigh the advantages which result from its administration. Further objectives are to reduce in number, diminish in severity, and/or otherwise relieve suffering from the symptoms of the disease in the individual in comparison to recognized standards of care. The invention may also be effective against neurodegenerative disorders in general: for example, dementia, depression, confusion, Creutzfeldt-Jakob disease, Huntington's disease, Parkinson's disease, loss of motor coordination, multiple sclerosis, stroke, and syncope. Thus, treatment may be directed at an individual who is affected or unaffected by the neurodegeneative disease.

Production of compounds according to present regulations will be regulated for good laboratory practices (GLP) and good manufacturing practices (GMP) by governmental agencies (e.g., U.S. Food and Drug Administration). This requires accurate and complete recordkeeping, as well as monitoring of QA/QC. Oversight of patient protocols by agencies and institutional panels is also envisioned to ensure that informed consent is obtained; safety, bioactivity, appropriate dosage, and efficacy of products are studied in phases; results are statistically significant; and ethical guidelines are followed. Similar oversight of protocols using animal models, as well as the use of toxic chemicals, and compliance with regulations is required.

The following examples substantiate the claims, inter alia, that brain microascular endothelium is dysregulated in Alzheimer's disease and gene expression profiling can be used as a prognostic indication for diagnosis and treatment. They are merely illustrative of the invention, and are not intended to restrict or otherwise limit its practice.

### EXAMPLES

Human Subjects

Microvascular brain endothelial cells (MBEC) are representative of the site of the blood-brain barrier. They were cultured from human brain tissue obtained at autopsy between 2.5 and 5 hrs postmortem. Average age of Alzheimer's disease (AD) cases was 75 years and that of age-matched control cases was 76 years. Equal numbers of male and female cases with and without disease were obtained. Thus, the observed changes are indicative of dementia, not age or gender. Cases are described in the Table. Total RNA was isolated from primary cultures of MBEC at passage 2-4.

TABLE
Description of Autopsy Cases

| Case | Diagnosis | Age | Gender | Passage No. |
|---|---|---|---|---|
| 1 | AD | 66 | M | 4 |
| 2 | AD | 86 | F | 2 |
| 3 | AD | 67 | F | 4 |
| 4 | AD | 79 | M | 3 |
| 5 | Control | 92 | F | 2 |
| 6 | Control | 88 | M | 4 |
| 7 | Control | 64 | M | 4 |
| 8 | Control | 59 | F | 4 |

Gene expression in MBEC was also compared between four young controls (e.g., less than about 40 years) and four aged controls (e.g., at least about 60 or 65 years). MBEC from young controls were collected from healthy individuals who died in motor vehicle accidents. There was one female of 37 years and three males of 21 years, 16 years, and 17 years. The average age of young controls was 23 years. The postmortem interval was again between 2.5 and 5 hrs. The aged controls were the same individuals who were used as age-matched controls in the comparison with Alzheimer's disease cases.

Neuropathological Analysis

Tissue blocks (1 cm³) from autopsy cases were fixed in 10% neutral-buffered formalin, pH 7.3 (Sigma), and embedded in paraffin or snap-frozen in liquid nitrogen-chilled isopentane. The tissue samples were obtained from the superior and middle frontal gyrus (Brodmann's areas 9 and 10) and cerebellar hemisphere. Tissue sections were stained with either hematoxylin and eosin (H&E) stain or thioflavin S by a modified Bielschowsky silver impregnation method (Gallyas stain). Thioflavin S stained sections were viewed through a Zeiss fluorescence microscope equipped with a narrow band, blue/violet filter from 400 nm to 455 nm. Two independent observers performed the examination. Diagnosis of Alzheimer's disease was made according to a modified CERAD (Consortium to Establish a Registry for Alzheimer's Disease) protocol (see Hyman and Trojanowski, J. Neuropathol. Exp. Neurol. 56:1095-1097, 1997).

Isolation and Culture of Human Microvascular Brain Endothelial Cells (MBEC)

MBEC were isolated postmortem from four Alzheimer's disease cases and four age-matched, nondemented controls using methods similar to those previously reported (Mackic et al., J. Clin. Invest., 102:734-743, 1998). Briefly, brain tissue was cut into small pieces, and then mechanically dissociated using a loose-fitting cell homogenizer in RPMI 1640 with 2% fetal calf serum (FCS) and penicillin/streptomycin (pen/strep). The homogenate was then fractionated over 15% dextran by centrifugation at 10,000 g for 10 min to obtain a brain microvessel pellet. Microvessels were further digested with 1 mg/ml collagenase/dispase and 5 μl/ml DNase in FCS-enriched medium for 1 hr at 37° C. Subsequently the cell suspension was centrifuged at 1,000 g for 5 min, and the cell pellet was plated on fibronectin-coated flasks in RPMI 1640 with 10% FCS, 10% Nuserum, endothelial cell growth factors, nonessential amino acids, vitamins, and pen/strep (Mackic et al., J. Clin. Invest., 102:734-743, 1998).

Characterization of Human Microvascular Brain Endothelial Cells (MBEC)

The P0 primary cultures were grown to confluence, and sorted based on LDL binding using the Dil-Ac-LDL method following the manufacturer's instructions (Biomedical Technology). Briefly, cells were incubated with Dil-Ac-LDL ligand for 4 hrs at 37° C., trypsinized, and then separated by fluorescence activated cell sorting (FACS). Labeled and unlabeled human umbilical vein endothelial cells (HUVEC) were used to set gating limits as positive and negative controls, respectively. Unlabeled MBEC were used to control for possible background staining or differences based on cell size. Positively sorted cells were plated on fibronectin- or collagen-coated flasks in the medium described above. Cultures were grown in 5% CO₂ and split 1:3 at confluency with collagenase/dispase (Mackic et al., J. Clin. Invest., 102:734-743, 1998).

Cryostat sections of the cortex adjacent to the isolation site of about 10 μm were air dried on slides (i.e., cryostat sections) or cultured MBEC were cytocentrifuged onto slides (i.e., cytospins). Cryostat sections or cytospins were characterized with a panel of cell-specific antibodies using single or double label staining. This panel included antibodies against Factor VIII or CD105 (endothelium), CD11b (monocyte/microglia), glial fibrillar acidic protein (astrocytes), α-actin (vascular smooth muscle), and neurofilament-α (neurons). Endothelial cells were greater than 98% positive for Factor VIII and CD105, but negative for the other markers of differentiated cells. By immunocytochemistry, a panel of antibodies including anti-Aβ₁₋₄₀, anti-Aβ₁₋₄₂, and others specific for the indicated gene products were used. Quantitation by such antibodies confirm the results obtained for differences in transcript abundance.

RNA Isolation from MBEC

About 5×10⁵ MBEC were plated in a 100 mm tissue culture dish. MBEC were cultured for 2-3 days until the monolayer was subconfluent (about 80%). Total RNA was isolated using TRIZOL reagent (Life Technologies) according to the manufacturer's instructions: cells were homogenized in a monophasic solution comprised of phenol and guanidine isothiocyanate, add chloroform and separate phases, differentially precipitate RNA, and wash and solubilize RNA (U.S. Pat. No. 5,346,994). Total RNA was visualized by gel electrophoresis and analyzed by spectrophotometry to assess the purity and integrity of the preparation.

Preparation of Labeled Target

Total RNA (10 μg) from each sample was used to generate high fidelity cDNA, which was modified at the 3′ end to contain an initiation site for T7 RNA polymerase following the manufacturer's instructions (SUPERCHOICE kit, Life Technologies). Upon completion of cDNA synthesis, 1 μg of product was used in an in vitro transripion (IVT) reaction that contained biotinylated UTP and CTP which were labeled for detection following hybridization to the array following the manufacturer's instructions (ENZO). Full-length IVT product (20 μg) was subsequently fragmented in 200 mM Tris-actetate (pH 8.1), 500 mM KOAc, and 150 mM MgOAc at 94° C. for 35 min. Following fragmentation, all components generated throughout the processing procedure (cDNA, full-length cRNA, and fragmented cRNA) were analyzed by gel electrophoresis to assess the appropriate size distribution prior to array hybridization.

High Density Oligonucleotide Array Hybridization

All samples represented were subjected to gene expression analysis with the Affymetrix HG-U95A high-density oligonucleotide array in the University of Rochester Microarray Core Facility. The HG-U95A array contains probe sets that correspond to information from 12,000 full-length cDNA from the Unigene cluster database. Each gene on the array is represented by 16-20 probe pairs of 25-mer oligonucleotides that span the gene's coding region. Each probe pair consists of a perfect match (PM) sequence that is complementary to the cRNA target and a mismatch (MM) sequence that has a single base pair mutation in a region critical for target hybridization. This sequence serves as a control for nonspecific hybridization. Hybridization, staining, and washing of arrays were performed in the fluidics station and hybridization oven (Affymetrix) following the manufacturer's instructions. Streptavidin phycoerythrin stain (Molecular Probes) was the florescent conjugate used to detect the targets hybridized to probe on the array. The detection and quantitation of target immobilized on the array were performed with a scanner (Hewlett Packard/Affymetrix) following the manufacturer's instructions. In addition, all arrays were scanned pre- and post-antibody amplification to address the possibility that the dynamic range of the scanner may be limiting.

All arrays used herein were assessed for “array performance” prior to data analysis. This process involves the statistical analysis of control transcripts that are spiked into the hybridization cocktail to assess performance. In addition, several genes have been identified on each array to assess the overall quality of signal intensity from all arrays. The results of these analyses have demonstrated that the performance of each array is within a small difference of each other at baseline. This analysis affords the necessary confidence needed to apply a global scaling approach to data normalization in subsequent analyses.

Data Analysis and Comparative Results

The Affymetrix data analysis suite was used to generate comparative results. Distinct algorithms were used to determine the absolute call which distinguishes the presence or absence of a transcript; the differential change in gene expression as measured by increase (I), decrease (D), marginal increase (MI), marginal decrease (MD), and no change (NC); and the magnitude of change which is represented as fold change. The mathematical definitions of each of these algorithms can be found in the Microarray Suite Analysis manual in the algorithm tutorial. In brief, the algorithm which defines the presence or absence of a gene takes into consideration the following qualitative and quantitative metrics from the raw data set: positive/negative ratio, positive fraction, and the log average ratio. The algorithm which defines the differential change in gene expression takes into consideration the following metrics from the raw data set: Max (Increase/Total, Decrease/Total), Increase/Decrease ratio, Log average ration change, and Dpos-Dneg ratio. The threshold setting for this decision matrix was set at default levels. Finally, the fold change calculation was based on the Average Difference of each probe set due to the fact that this output is directly related to its expression level.

The fold change of any transcript between the baseline and experimental was calculated following global scaling. All arrays within this data set were normalized by global scaling (target intensity=2500). Super Scoring (SDT=3) was applied to all probe sets of eight probe pairs or more, which means that any probe pair average difference that exceeded 3 standard deviations of the mean of all probe sets was not calculated in the Average Difference metric.

Pairwise comparisons were made between groups of different Alzheimer's disease patients and age-matched, nondemented controls, and aged-matched controls and young controls. Each group analyzed consisted of samples from four independent primary culture cell lines. Pairwise comparisons were performed and all 16 possible comparisons were analyzed. A limit of 2.5-fold increase or decrease was imposed on the original analysis of all comparisons. The data is represented and sorted by “hit number” which represents the number of times a significant difference of gene expression was noted in the comparisons meeting this 2.5-fold criteria. The limit for including significant changes was made at 10 out of 16 possible hits to limit the possibility of a biased contribution by any given sample. The data is represented as average fold difference.

When a 2.5-fold change was set as the limit, about 4400 of the about 12,000 genes on the HG-U95A array (about 39%) showed at least this amount of increase or decrease. Results with selected genes are discussed below. Although GENECHIP technology was used here, similar results are expected if other array technology was used such as spotted arrays (Affymetrix, Incyte Genomics) or printed arrays (Rosetta Inpharmatics). Moreover, differential display (U.S. Pat. No. 5,665,547); serial analysis of gene expression (U.S. Pat. No. 5,866,330, Genyzme); bead arrays analyzed by fiber optics (WO 98/50782, Illumina) or sorting (U.S. Pat. No. 6,265,163, Lynx) are expected to arrive at similar results. Similarly, biosensors which detect protein (U.S. Pat. No. 6,329,209) or products on a cell surface (U.S. Pat. No. 6,210,910) can be used for gene expression profiling.

Differences in Gene Expression Profiles

Without limiting the significance of any of the about 39% of genes detectable with the HG-U95A array, particular members are discussed below with attention to what they teach about the etiology and biology of Alzheimer's disease. For the genes which were assayed by more than one gene profiling procedure, arrays gave results similar to those obtained by RT-PCR of transcripts and proteomic studies of proteins (i.e., the direction of the change was identical but the magnitude of the change might not be comparable), but array signals were more convenient to quantitate.

The MBEC from brains of control cases (i.e., normally aged individuals) produce several neurotrophic factors and related proteins, as well as growth factor binding proteins, that are important for neuronal survival and the maintenance of differentiated phenotypes in the aging brain. In contrast, the MBEC from brains of individuals with Alzheimer's disease highly down regulate expression of these genes and therefore are unable to offer neuronal support. This leaves neurons without clear guidance about how to survive and to maintain their highly differentiated phenotype which is necessary for normal function. Loss of this support may predispose to neuronal injury and loss, and then dementia in Alzheimer's disease. It may also lead to their reentering the cell cycle in response to carcinogens and other stimulants of cell growth and division (see, for example, discussions below of loss of detoxification function of the blood-brain barrier and endothelium growth dysregulation).

MBEC of normal, elderly humans express neurotrophic factors and related proteins such as, for example, nerve growth factor (NGF), brain-derived neurotrophic factor (BDNF), bone morphogenetic protein-1 (BMP-1), fibroblast growth factor-5 (FGF-5), fibroblast activation protein-α (FAP-α), and intercrine-α (IRH). They also produce different growth factor binding proteins that regulate transport of those growth factors in brain extracellular fluids and therefore may indirectly influence neuronal function, such as, for example, insulin-like growth factor binding protein-1 (IGFBP-1) and insulin-like growth factor binding protein-5 (IGFBP-5).

In MBEC of patients with Alzheimer's disease, expression of these important genes was significantly down regulated. This may predispose to neuronal injury and loss, and then unsuccessful aging as shown by dementia because of the withdrawal of their neurotrophic or related functions. The gene's accession number and function are shown in parentheses. NGF (M57399, neuronal survival and differentiation) was 17-fold decreased. FGF-5 (M37825, putative neurotrophic factor) was decreased 6-fold. BDNF (M61176, neurotrophic factor) was 4-fold decreased. BMP-1 (M22488, putative trophic factor involved in NGF processing) was 11-fold decreased. FAP-α (U09278, tissue remodeling and repair) was 16-fold decreased. IRH (U19495, B-cell growth stimulating factor and putative trophic factor) was 10-fold decreased. IGFBP-5 (L27560, IGF transport protein) was 9-fold decreased. IGFBP-1 (M74587, IGF transport protein) was 8-fold decreased.

It is suggested that loss of function or significantly diminished function of several MBEC genes that encode for neurotrophic factors and related proteins is responsible for the development of Alzheimer's disease, its pathology, and neuronal loss. These novel findings suggest that Alzheimer's disease may be primarily a vascular disease resulting from a failure of MBEC to produce neurotrophic factors or related proteins necessary for neuronal survival, maintenance of their differentiated phenotypes, and their normal functioning, all of the preceding may be a prerequisite for healthy mental status. Failure of MBEC in Alzheimer's disease to produce neurotrophic factors and related proteins leads to increased neuronal susceptibility to cellular stress, injury, and may ultimately result in neuronal cell death.

MBEC of brains from Alzheimer's disease patients are also unable to protect the brain from effects of circulating toxins, brain-derived metabolic waste products, and other neuroactive substances. The changes observed in MBEC gene expression of Alzheimer's disease cases suggest that there is malfunction of major detoxification enzymatic pathways at the blood-brain barrier, enhanced potential for generation of neurotoxins and carcinogens, and down regulation of major efflux transport systems at the blood-brain barrier which normally protect against potentially damaging neuroactive substances in brain at a low level. These changes may ultimately lead to neurovascular uncoupling, and then result in loss of neuronal synaptic activities and neuronal death.

MBEC form the blood-brain barrier in vivo that prevents toxins from entering the brain. The blood-brain barrier also regulates the composition of brain extracellular fluid to be optimal for neuronal functioning. The barrier between the blood and the brain is in part represented by the so-called “enzymatic” barrier. In addition, several transport proteins at the blood-brain barrier remove metabolic waste products from brain into the blood that include potentially damaging neuroactive excitatory molecules and possibly macromolecular aggregates as seen in Alzheimer's disease. Furthermore, MBEC form tight junctions in vivo with a zipper-like continuous cellular membrane which forms the basis of the so-called “anatomical” blood-brain barrier. Extracellular matrix proteins contribute to the basement membrane and the tight and adherens junctions at the blood-brain barrier. The latter barriers limit free exchange of solutes between blood and brain.

Anatomic and enzymatic components of the blood-brain barrier are reviewed in McComb and Zlokovic (2000) Cerebrospinal fluid and the blood-brain interface, In: Textbook of Pediatric Neurosurgery. W.B. Saunders, Philadelphia.

Several genes encoding important detoxifying enzymes were expressed in the MBEC of nondemented, normally-aged individuals. These genes tend to prevent circulating neurotoxins and neuroactive substances to penetrate the brain and therefore are neuroprotective. Genes were expressed for transport proteins which could maintain low levels of excitatory neurotransmitter in brain and possibly also remove potentially toxic macromolecular aggregates from brain. The presence of several genes encoding for mature extracellular matrix proteins and cytoskeletal proteins that may be associated with adherens junctions of the blood-brain barrier were also detected.

But in age-matched Alzheimer's disease patients, MBEC revealed significant changes in the expression of detoxifying genes. This suggests a failure of MBEC of brains from Alzheimer's disease patients to protect neurons from peripheral toxins and the possibility that such MBEC may be directly toxic to neurons by converting circulating “protoxicants” (i.e., molecules that are precursors of toxins) into neurotoxins. Expression of genes encoding transport proteins that mediate brain to blood transport of excitatory neurotransmitters and macromolecular aggregates that accumulate in Alzheimer's disease were markedly down regulated. MBEC of brains from Alzheimer's disease patients also cannot produce mature extracellular matrix molecules. This may further impact the integrity of the blood-brain barrier by favoring formation of an immature basement membrane. In addition, a possible loss of appropriate relationship between the proteins that link the cytoskeleton with the adherens and/or the tight junctions may increase the vulnerability of tight junction complexes.

MBEC of normal, elderly humans express several genes encoding detoxifying enzymes such as, for example, dihidrodiol dehydrogenase (DDH) and dioxin-inducible cytochrome P450 (CYP1B1). DDH is a member of the monomeric oxidoreductase gene family and can metabolize steroids, polyols, prostaglandins, and proximate dihidrodiol polyaromatic hydrocarbon carcinogens. All these reactions have important metabolic consequences in either elimination or detoxification of these compounds. It also may tend to reduce the levels of brain-derived neuroactive peptides that may diffuse away from synapses to the blood-brain barrier where they are further inactivated to smaller degradation products prior to the transport into the blood. CYP1B1 detoxifies many neuroactive drugs and toxic pollutants (e.g., dioxin is a prototype of a large class of halogenated aromatic hydrocarbons). Collectively, the activities of these enzymes provide a significant protective enzymatic barrier that can play an important role in detoxifying the brain during normal aging. Therefore, MBEC-derived enzymes are indirectly neuroprotective in the aging brain.

Alzheimer's disease is associated with a greater risk of neuronal injury that could lead to neuronal loss and neuronal cell death due to the failure of detoxifying enzymes of the MBEC to influence the neuronal environment. A remarkable shut down of the following MBEC genes was observed in Alzheimer's disease: DDH (U05861) was 16-fold decreased and CYP1B1 (U03688) was 24-fold decreased. A significant reduction in expression of the DDH and CYP1B1 genes in MBEC of brains from Alzheimer's disease patients may result in an accumulation of toxins. When DDH activity derived from MBEC is shutdown, increases in levels of steroid hormones, polyol alcohols that are associated with brain edema, prostaglandins associated with changes in blood flow, and proximate dihidrodiol polyaromatic hydrocarbon carcinogens are expected in brain. In addition to injury, several teratogenic substances or “carcinogens” may stimulate neurons to enter the cell cycle (as observed in Alzheimer's disease) and ultimately result in neuronal death. Accumulation of drugs, xenobiotics, and toxic pollutants (including carcinogenic and teratogenic hydrocarbons) can be expected with the shutdown of MBEC-derived CYP1B1 activity. This can also stimulate neurons to enter the cell cycle, which they fail to complete, and then die (i.e., apoptosis).

During normal aging in nondemented humans, MBEC contain relatively low levels of N-methyltransferase (NNMT). NNMT catalyzes the N-methylation of nicotinamide and other pyridines to form pyridinium ions. Several N-methylpyridinium compounds are toxic: for example, paraquat or the neurotoxin 1-methyl-4-phenylpiridinium ion, a metabolite of 1-methyl-4-phenyl-1,2,3,6,-tetrahydropyridine. Selected pyridine substrates may function as “protoxicants” in the brain. NNMT activity could have implications for individual differences in xenobiotic and drug toxicity. Thus, low levels of expression in MBEC as observed in age-matched nondemented individuals would reduce the opportunity to generate potentially toxic compounds. In contrast, higher levels of NNMT in MBEC of brains from Alzheimer's disease patients could be associated with or may be caused by the greater risk of neurotoxin production.

In brains from patients with Alzheimer's disease, gene expression in MBEC of NNMT (U08021) is 17-fold increased. This result suggests that MBEC may represent an important source for neurotoxins in Alzheimer's disease. These neurotoxins may be generated from many different “protoxicant” precursor molecules that are taken up from the circulation at the blood-brain barrier and could be converted into toxins.

MBEC of brains from nondemented, normally-aged individuals express genes encoding for glutamate transporters, neuronal pentraxin-1, multidrug resistance protein-1 (MRP-1), ATP-binding cassette transporter 1 (ABCA1), and low density lipoprotein receptor-related protein-1 (LRP-1). Glutamate transporter, also known as shared glutamate/aspartate transport system, has been described at the blood-brain barrier. Its primary role is to transport excess neurotansmitter glutamate into the blood upon its release from presynaptic vesicles into the extracellular fluid. Neuronal pentraxin-1 (NPTX-1) typically mediates uptake of macromolecules and other extracellular material into synaptic vesicles. It is present in MBEC of brains from normally-aged individuals, where it is possible that it serves the same function (i.e., to adsorb and eliminate potentially toxic macromolecular aggregates from the extracellular fluid). MRP-1 removes xenobiotics, steroid hormones, and bile salts from the brain; ABCA1 and LRP-1 remove cholesterol from the brain.

In brains from patients with Alzheimer's disease, gene expression in MBEC of transport/pump genes are highly down regulated: glutamate transporters (U08989 and A1928365) by 10-fold and 12-fold, respectively; NPTX-1 (U61849) by 17-fold; MRP-1 (AF022853) by 5-fold; ABCA1 (AB020629) by 15-fold; and LRP-1 (X13916) by 3-fold. Down regulation of glutamate transporters at the blood-brain barrier in Alzheimer's diseases may lead to accumulation of glutamate in brain. Glutamate is often released during brain hypoperfusion and reduced blood flow as described in Alzheimer's disease patients. Impaired glutamate efflux from the brain may critically increase its concentrations and could be neurotoxic due to excitotoxicity, which ultimately leads to neuronal injury and cell death. Although the function of NPTX-1 at the blood-brain barrier is still not fully understood, it is envisioned that this transporter may be involved in removing the macromolecules from brain extracellular space that accumulate during normal aging and particularly in Alzheimer's disease. Thus, its shutdown may favor amyloid accumulation in brain. Similarly, down regulation of MRP-1, ABCA1 and LRP-1 may lead to accumulation of xenobiotics, neurotoxins, and cholesterol in brain.

There was also widespread and marked down regulation in MBEC from patients with Alzheimer's disease of genes which encode extracellular matrix proteins. Elastin (X52896) was 68-fold decreased. Autotaxin (L35594) was 49-fold decreased. Different types of collagen were down regulated: collagen VIα2 (M20777) was 68-fold decreased, collagen VIα3 (X52022) was 18-fold decreased, collagen VIIIα1 (X57527) was 15-fold decreased, and collagen XIIIα1 (M59217) was 3-fold decreased. Cadherin 6 (D31784) was 18-fold decreased. Laminin α4 (S78569) was 25-fold decreased and laminin M (Z26653) was 5-fold decreased. Integrin α7 (AF032108) was 68-fold decreased and integrin β4 (X53587) was 14-fold decreased.

Cytoskeletal proteins were affected as well in Alzheimer's disease. Cytoskeletal proteins that may be involved in control of the cell shape and connection with proteins in the adherens junctions such as, for example, smooth muscle myosin heavy chain (AF013570), myosin light chain kinase (U48959), and smooth muscle γ-actin (D00654) were 55-fold decreased, 39-fold decreased, and 132-fold decreased, respectively. Moreover, immature intracellular filaments such as keratin 18 (M26326) and sarcolectin (AJ238241) were increased by 28-fold and 5-fold, respectively.

These data suggest that the integrity of the blood-brain barrier may have been compromised in Alzheimer's disease because of an incompetent basement membrane and loss of normal cell shape as a result of loss of important cytoskeletal proteins and gain of immature and incompetent cellular filament function. As a result, the tight junction complex that is the basis of the anatomical blood-brain barrier could be more vulnerable in Alzheimer's disease. These changes may lead to uncontrolled exchange of solutes between blood and brain that may reinforce the accumulation of toxins in brain.

Based on gene expression profiling, it is suggested that loss of function and/or significantly diminished function of several MBEC genes that encode for detoxifying enzymes may contribute to the development of Alzheimer's disease. Compromised integrity of the anatomical blood-brain barrier will promote neuronal injury by letting toxins and carcinogens enter the brain. Vulnerable tight junctions will reinforce failure in the blood-brain barrier. Thus, changes in the blood-brain barrier (e.g., at least some MBEC) in Alzheimer's disease may produce neurotoxins when challenged by “protoxicants” and pollutants. Neuronal cell death may result from neurointoxication (e.g., overflow of the excitatory neurotransmitters and xenobiotics) and/or forcing neurons to enter the cell cycle by teratogenic and carcinogenic stimuli. Once the neurons enter the cell cycle, they will die because they are incompetent to follow through to completion. A strategy that will replenish the blood-brain barrier to accomplish its goal of detoxification and enhance its anatomical and enzymatic integrity promises to inhibit or slow neurodegeneration. Thus, MBEC of the blood-brain barrier are provided as a major therapeutic target in Alzheimer's disease.

MBEC of brains from Alzheimer's disease patients display a pattern of gene expression consistent with marked growth dysregulation similar to transformed cells. Endothelium, unlike virtually all other terminally differentiated tissues, does not undergo malignant transformation that results in cancer. This discovery suggests that a large fraction of Alzheimer's disease is due to fundamental growth dysregulation of MBEC. A multi-step process is envisioned whereby inhibition of the activity of growth suppressors and activation of the activity of growth promoters in the vascular system produce clinically recognizable Alzheimer's disease. Growth dysregulation in MBEC is demonstrated by pronounced down regulation of tumor suppressor genes, up regulation of cell cycle genes, and large-scale down regulation of extracellular matrix genes, a pattern similar to events occurring during oncogenesis. But most importantly, direct measurements of growth rates of MBEC primary cultures revealed that doubling times were slowed in Alzheimer's disease, possibly due to suicidal cell death by apoptosis, because MBEC are unable to successfully complete the cell cycle. These results indicate that Alzheimer's disease is a disease of unbalanced and incomplete MBEC growth resulting in aberrant function of brain microvasculature.

Growth dysregulation requires down regulation of genes that inhibit growth. In MBEC of brains from Alzheimer's disease patients, but not in age-matched controls, several growth suppression genes were robustly decreased. The product of growth arrest gene-1 (GAS1) inhibits unrestrained mitotic activity and the gene (L13698) was decreased by 38-fold. The growth suppressing function of GAS-1 requires p53 and pRB. The similarly acting GAS-1a gene (L13698) was decreased by 29-fold. β,γ-crystallin family member AIM1 is associated with suppression of malignant melanoma and the gene (AI800499) was decreased by 6-fold. MN1 (X82209), a putative tumor suppressor gene in malignant meningioma that is inactivated by translocation, was decreased by 11-fold. The interferon-inducible protein 9-27, involved in transduction of antiproliferative signals, was decreased by 33-fold (J04164). Aminopeptidase N or CD13 (M22324) was 25-fold decreased. Ubiquitin C-terminal hydrolase, a putative tumor suppressor gene, was decreased by 41-fold.

Moreover, increased activity of the mitotic cell cycle is another characteristic of rapidly dividing tissues and transformed cells. In MBEC obtained from brains of patients with Alzheimer's disease, expression of genes encoding cyclins and CDC kinases were increased relative to controls. Both cyclin B1 (M25753) and cyclin B2 (AL080146) genes were increased by 4-fold and 18-fold, respectively. Cyclins B1 and B2 promote progression of cell into M phase. Cyclin B1 activates Cdc2, a kinase that stimulates cell progression into M phase. BUB1 (AF053305), which encodes a mitotic checkpoint control kinase that functions in spindle checkpoint control, was increased by 4-fold. P55CDC (U05340), which encodes a homolog of yeast Cdc4 that promotes progression into anaphase, was increased by 25-fold. KAP (L25876), which encodes a kinase-associated kinase that regulates of Cdc2 activity and the G2/M transition, was increased by 5-fold. CDC2 (X05360) and CDC2A (M68520), which encode kinases that promote progression into M phase, were increased by 2-fold and 10-fold, respectively. In addition, a number of genes involved in chromosome processing, segregation, and assembly are dysregulated. Mitotic kinesin-like protein-1 and kinesin-like spindle protein are motor enzymes required for mitotic progression and they promote segregation of chromosomes during cell division (cytokinesis). Genes (X67155 and U37426, respectively) encoding those motor enzymes were increased by 7-fold and 3-fold, respectively. Also, chromosome segregation gene (AF053641), chromosome condensation-related protein (D63880), serine/threonine kinase BTAK (AF011468), and topoisomerase II (A1375913) were increased by 3-fold, 15-fold, 5-fold, and 3-fold, respectively. G0/G1 switch gene-2 (M69199) was increased by 39-fold.

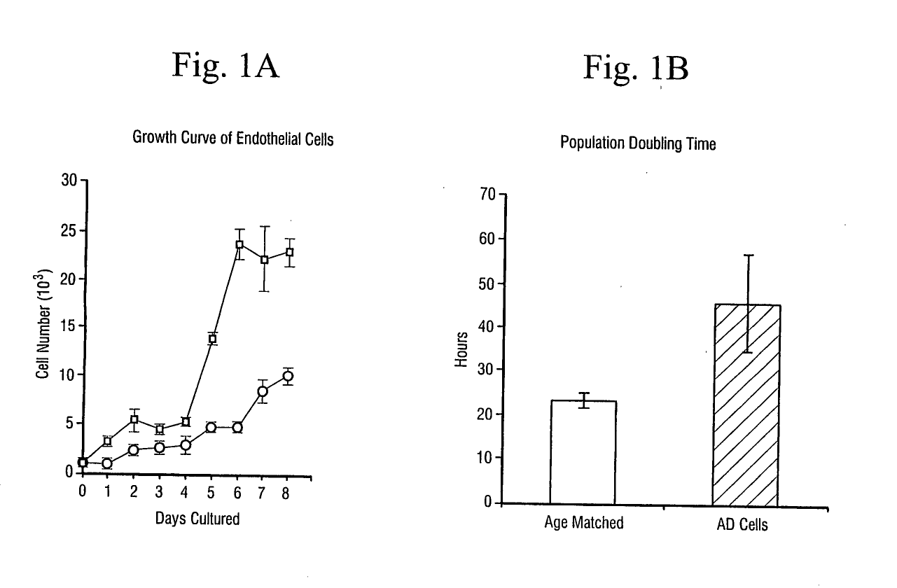

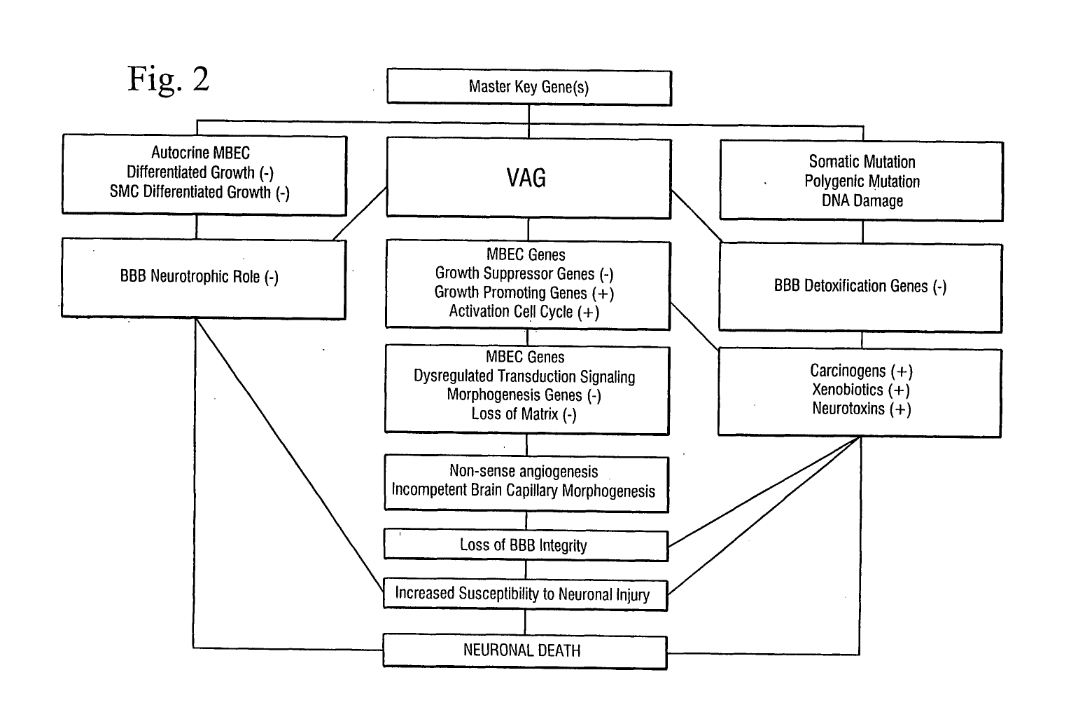

Growth kinetics of MBEC primary cultures were compared between brains from Alzheimer's disease patients and age-matched controls (FIG. 1A). The population doubling time of Alzheimer's disease MBEC was 2-fold greater than controls (FIG. 1B). This apparent contradiction between the slowing of cell doubling time and the increased expression of genes associated with cell growth and division could be explained by the inability of Alzheimer's disease MBEC to successfully complete the cell cycle. These cells could be arrested either in G2/M phase and/or in G0/G1 or S phase. If MBEC are unable to complete the cell cycle, it is likely they will commit suicide (i.e., apoptosis) and die. Therefore, it is suggested that the growth dysregulation measured directly by population analyses indicates incomplete completion of mitosis and cytokinesis. The defect in Alzheimer's disease MBEC therefore may be due to unbalanced growth stimulation and perturbation of their cellular properties.

It is envisioned that profound growth dysregulation of MBEC is involved in the etiology of Alzheimer's disease. By gene expression profiling, decreased expression of growth suppressing genes, increased expression of growth promoting genes, and the general activation of the cell cycle were observed. This MBEC molecular phenotype contrasts with the cell phenotype of slowed cell doubling and overall growth. These data suggest that MBEC in brains of Alzheimer's disease patients are stimulated to express growth promoting molecules but cannot complete mitosis, and therefore may chose a pathway that leads to apoptosis and cell death. Incomplete cytokinesis can account for altered properties of the Alzheimer's disease microvasculature.

Increased expression of growth promoting genes is a characteristic of rapidly dividing tissues and transformed cells. In MBEC of brains from Alzheimer's disease patients, there were several genes encoding growth stimulatory proteins that were increased: VEGF-C (X94216) by 4-fold; VEGF-related protein (U43143) by 13-fold; receptor tyrosine kinase DTK (U18934), which encodes a receptor for a putative mitogen of MBEC, by 8-fold; β-thromboglobulin (X54489), which stimulates the growth of melanoma, by 13-fold; tissue factor (J02931), which in addition to its clotting function also stimulates tumor growth and angiogenesis, by 7-fold; and aryl hydrocarbon receptor nuclear translocator-2 or ARNT2 (AB002305), which induces expression of VEGFR, angiopoetin-1, and tie-2, by 4-fold. Other related genes are the protooncogene Wnt-5 (L20861), which encodes a putative endothelial growth stimulating factor, and a mesoderm-specific transcript (D78611), which encodes an angiogenesis related protein, that were both increased by 3-fold.

In contrast, expression of genes that may be involved in regulation of capillary morphogenesis was decreased: Notch-3 (U97669), which is involved in extension and stabilization of vascular networks, by 10-fold and Semaphorin-III (L26081), which is a putative capillary morphogenesis factor, by 9-fold.

Several factors with possible involvement in the autocrine regulation of MBEC differentiated growth were significantly decreased by 4 to 17-fold: FGF-5, NGF, BDNF, BMP-1, FAP-α, IGFBP-5, IGFBP-1, IRH, and angiotensin receptor-II (S77410). Taking into account that production of extarcellular matrix is significantly down regulated in MBEC of brains from Alzheimer's disease patients, it may be concluded that cellular dysregulation of MBEC may lead to nonsense angiogenesis and incompetent capillary morphogenesis with loss of blood-brain barrier integrity and normal functioning.

There was significant down regulation of several trophic factors as explained above (i.e., NGF, FGF-5, BDNF, BMP-1, FAP-α, IGFBP-5, IGFBP-1, and IRH). They may negatively impact on vascular smooth muscle cells (SMC) that would possibly need this trophic support from the endothelium to maintain their differentiated phenotype. This can lead to SMC degeneration and loss of support for vascular SMC, which in turn can be associated with dysregulation of the blood flow and increased risk for hemorrhage, as observed in AD patients with cerebral amyloid angiopathy (CAA).

Protein synthesis and processing are markedly affected in MBEC of brains from Alzheimer's disease patients. For example, the gene (M58459) for the ribosomal protein RPS4Y linked to the Y chromosome was decreased by 32-fold, while transglutaminase, a cross-linking enzyme of proteins, was increased by 85-fold. Energy metabolism in MBEC of brains from Alzheimer's disease patients is reduced at least in part due to down regulation of gene expression of mitochondrial citrate transport protein (X96924) by 24-fold and creatine kinase (L26336) by 13-fold.

Gene expression of signaling molecules is also changed in MBEC of brains from Alzheimer's disease patients. CL100 MAP kinase phosphatase inactivates phosphorylated MAP kinase (MAPK) and the gene (X68277) was decreased by 48-fold. The RGS7 regulator of G-protein signaling protein (U32439), a GTPase activating protein, and Rab-GAP/TBC containing protein (AB024057), a putative GTPase activating protein, were both decreased by 5-fold. These changes may indicate loss of tight regulation of signal transduction pathways. In particular, MAPK may remain in active phosphorylated form that may represent constant signal possibly linked to cell proliferation. Down regulation of GTPase activating proteins may cause similar problems in intracellular signaling. Interferon-inducible protein 9-27, part of a membrane complex relaying growth inhibitory signals, was decreased by 33-fold; this may be a stimulus for the uncontrolled proliferation of MBEC of brains from Alzheimer's disease patients. Two other genes encoding signal transduction regulatory molecules, Arg/Abl interacting protein involved in assembling signaling complexes (AF049884) and glutamic acid rich protein (GARP) involved in protein-protein interactions in signal transduction (Z24680) were decreased by 11-fold and 22-fold, respectively.

The increase in gene expression of tissue factor by 7-fold in MBEC of brains from Alzheimer's disease patients may create a hypercoagulable status in the brain microcirculation of patients. Decay accelerating factor (M31516), an inhibitor of the complement cascade, and complement factor H (M65292) were decreased by 3-fold and 17-fold, respectively. It is envisioned using activated protein C (APC) as a natural anticoagulant for treatment. In addition to its beneficial anticoagulation effects, APC may also be useful to alleviate cellular stress and may be neuroprotective in Alzheimer's disease patients.

The functions discussed for MBEC may act independently, additively, or synergistically in Alzheimer's disease: loss of neurotrophic support, reduced detoxification, dysregulation of cell growth in the microvasculature (e.g., smooth muscle, endothelial cell) leading to nonsense angiogenesis, and incompetent capillary morphogenesis. This discovery shifts attention from plaque formation in the neuronal and vascular compartments to the microvasculature that comprises the blood-brain barrier in understanding Alzheimer's disease. It is envisioned that these pathways may be coordinated by master key genes which regulate one or more of the pathways, and may even be involved in feedback regulation by the products of those pathways (see FIG. 2).

Twenty-three genes expressed in the MBEC of nondemented, elderly humans were discovered and termed vascular aging genes (VAG), which were involved in: cell cycle regulation [e.g., p16 inhibitor of G1 cyclin/cdk enzymes, absent in melanoma 1 (AIM1), growth arrest-specific 1 (GAS1)]; differentiation [e.g., aminopeptidase N (CD13), aryl hydrocarbon receptor nuclear translocator 2 (ARNT2), DTK receptor tyrosine kinase, ephrin B2, growth arrest-specific homeobox (GAX), Notch-3, semaphorin III]; extracellular matrix [e.g., collagen VIα, elastin, integrin α7, integrin β4]; toxin metabolism [e.g., dihydrodiol dehydrogenase (DDH), dioxin-inducible cytochrome P450 (CYP1B1), N-methyltransferase (NNMT)]; membrane transport [e.g., ATP-binding cassette transporter 1 (ABCA1), glutamate transporters, low density lipoprotein receptor-related protein-1 (LRP-1), multidrug resistance protein-1 (MRP-1)]; and cell growth and support [e.g., brain derived neurotrophic factor (BDNF), nerve growth factor (NGF)]. VAG may be important for successful aging of the nervous system, neuronal survival, and the maintenance of differentiated phenotypes. One or more of the VAG may also act as a “master key” gene important for the nondemented mental status of successfully aged individuals.

The above-described gene expression profiling was repeated with young controls to determine whether changes would be observed due to aging. These control groups, young and normally-aged, were not diagnosed with Alzheimer's disease.

Expression of the neurotrophic genes discussed above was confirmed in MBEC from young controls. Therefore, these genes may be important for normal functioning in young brains and they do not appear to be expressed only in the old as an adaptation to a more demanding and challenging brain environment. In contrast, there was no change in the gene expression of nerve growth factor (NGF), fibroblast growth factor (FGF-5), brain derived neurotrophic factor (BDNF), bone morphogenetic protein-1 (BMP-1), fibroblast activation protein (FAP-α), insulin-like growth factor binding protein-1 (IGFBP-1) and intercrine-α between young and normally-aged controls. Thus, MBEC from the young produce neurotrophic factors that are important for both neuronal survival and maintenance of their differentiated phenotypes. There was no change in the expression of genes encoding for neurotrophic factors with normal aging associated with normal mental status.

Detoxification enzymes are normally present in young MBEC, and two were up regulated in normally-aged controls (i.e., DDH increased by 8-fold and CYP1B1 increased by 2-fold). This suggests that the blood-brain barrier increases its capability to degrade potential carcinogens, drugs, steroids, prostaglandinds, and polyols with normal aging. No change was observed in NNMT, an enzyme involved in production of neurotoxins. There was also no change in MBEC gene expression of efflux transporters, such as glutamate, MRP-1, or ABCA1. There was, however, an increase in the expression of NPTX-1, pentraxin, a receptor taught to be involved in the clearance of macromolecules from brain extracellular fluid. This may represent, again, an adaptation in older age associated with an increased capability of the blood-brain barrier to remove “waste” macromolecules.

No significant changes (i.e., cut-off greater than 2.5-fold) in tumor suppression genes was observed between normally-aged and young MBEC. GAS1, GAS1a, AIM-1, MN-1, and ubiquitin C-terminal hydrolase were not significantly different. An exception was a decrease by 27-fold interferon-inducible protein 9-27, which is involved in the transduction of antiproliferative signals. These findings are consistent with previous reports for normally-aged fibroblasts and progeria fibroblasts when no changes in general were observed in tumor suppressor genes during normal or accelerated aging. Occasional exceptions were observed such as down regulation in BRAC-1 associated RING domain isolated from progeria, a form of skin disease with accelerated aging (Ly et al., Science 287:2486-2492, 2000).

The direction of changes observed in some genes regulating the cell cycle in normally-aged MBEC as compared to young MBEC were generally consistent with previous reports describing changes in genes controlling cell cycle and chromosome processing and assembly at an older age in different cell types. However, changes in normally-aged MBEC were much less pronounced than previously reported changes in this functional group of genes (ibid.). For example, no changes were observed in cyclins B1 and B2, CDC2A, KAP, Bub1, chromosome segregation gene, chromosome condensation-related gene, topoisomerase II, and mitotic kinesin-like protein-1. But down regulation was observed with some genes involved in the control of cell progression through G2/M phase, such as a 3-fold decrease of CDC2, a 22-fold decrease of p55CDC, and 3-4-fold changes in genes that are involved in chromosomal segregation (RB-associated protein HEC), centrosome-associated kinase (BTAK), and kinesin-like spindle protein. These changes may suggest that MBEC from older subjects may be at an increased risk for mitotic misregulation, but the number of genes affected is still relatively small comparing with older fibroblasts and progeria fibroblasts. Although changes in the cell cycle genes and genes involved in chromosomal processing and assembly may indicate the possibility for a risk for increased rate of somatic mutation, leading to numerical and structural chromosomal aberrations and mutations that manifest as an aging phenotype, compensatory changes were also observed to balance the potentially altered cell cycle (e.g., a 7-fold increase in GOS2).

Aberrant regulation of some genes controlling the cell cycle and chromosomal processing were observed in normally-aged MBEC comparing to young MBEC, which suggests an aged phenotype. But the changes were not as pronounced as in other aged cell types (e.g., aged fibroblasts, progeria fibroblasts). This may reflect the relative capability of MBEC from normally-aged individuals to control their cell cycle and phenotypes. The cell cycle may still be preserved, but is somewhat slower as shown by their growth curves. The population doubling time of young controls was 21.5 hr±2.76 hr (n=4), which is not significantly different from aged controls.

It is important to note that changes in the expression of genes controlling the cell cycle and chromosomal processing in normally-aged MBEC are generally in a direction opposing those changes observed in MBEC from Alzheimer's disease patients. This emphasizes even more that dysgenesis and dysregulation of MBEC from Alzheimer's disease patients are very specific for this disease, and do not share common features with normal aging.

In contrast to Alzheimer's disease, where there is a significant loss of genes encoding the extracellular matrix proteins, most of these genes were not changed in normally-aged MBEC as compared to young controls: elastin, collagen VIα2, collagen VIα3, collagen XIIIα1, cadherin 6, laminin M, integrin α7, and integrin β4. A few genes were, however, up regulated in normally-aged MBEC such as autotaxin, involved in cellular chemotaxis; and collagen VIIIα1 and laminin M. Up regulation of one collagen and one laminin gene may be related to potential thickening of the basal membrane observed in normally-aged individuals. Again, these changes were less pronounced than in normally-aged fibroblasts and progeria fibroblasts where the larger number of extracellular matrix genes are affected. Several genes encoding enzymes that degrade extracellular matrix proteins are increased in aged fibroblasts and progeria fibroblasts suggesting degradation of skin matrix. This is corroborated by degenerative changes in the skin in normally-aged individuals and, in particular, in patients with progeria seen clinically. No similar changes were observed in normally-aged MBEC, which may indicate that their aging phenotype is much closer to that found in young individuals. This suggests that MBEC in the normally-aged brain may be good candidates for perfect genomic match, as seen in younger individuals, assuming that neurovascular match in young subjects is ideal.

Regarding the cytoskeletal proteins, the genes that are potentially involved in the regulation of the adherens junctions and endothelial shape, gene expression in normally-aged MBEC of smooth muscle γ-actin and myosin light chain kinase was increased by 5-fold and 46-fold, respectively. This may suggest compensatory changes in these cells to resist potential hypoperfusion due to lesser metabolic brain demand, as it happens in normally-aged brains. Hypoxic cells may lose their cytoskeletal proteins and acquire bizarre shapes, as recently shown with bovine MBEC, that may only make changes in the blood flow more profound in the aging brain. Thus, keeping a tight shape may offset this potential risk.

Most endothelial growth promoting factors were not changed with aging: VEGF-C, Wnt-5, DTK, and β-thromboglobulin offer relatively good support for growth. But some exceptions are VRP, tissue factor, and ANRT2, which were decreased by 14-fold, 3-fold, and 3-fold, respectively. This may put at risk the capability of these cells to grow, similar to occasional changes in growth promoting factor genes in aged cells in general (e.g., fibroblasts). Importantly, no changes in capillary morphogenesis genes (e.g., Notch-3 and semaphorin III) were observed. Also preserved was the potential for autocrine regulation of MBEC differentiated growth.

An exception to this observation was an increase in expression of IGFBP-5 in MBEC from normally-aged controls, a gene that encodes for transport protein for IGF. This could be explained by envisioning a compensatory response due to a higher demand for insulin-like growth factor in the normally-aged brain vs. young brain. It is noteworthy that genes described in this category may help in maintaining differentiated phenotypes of MBEC via autocrine regulation, and vascular smooth muscle cells via paracrine regulation as described below.

The same trophic factors that may support neuronal survival and differentiated phenotype and autocrine regulation of differentiated MBEC may also be important for vascular SMC, and are not altered by normal aging. This is consistent with preservation of autoregulation of the cerebral blood flow and retention of the smooth muscle layer in the vascular system in normally-aged brains.

There was an increase by 32-fold in the gene encoding ribosomal protein RPS4Y linked to the Y chromosome. This is compatible with the belief that increased protein synthesis may be important to offset cell cycle genes that were down regulated. On the other hand, a degree of metabolic failure, which was reflected in a decrease by 53-fold down for mitochondrial citrate transport protein is consistent with the concept of age-related mitochondrial dysfunction, as previously shown for other cell types.

Consistent with the concept that MBECs in normally aged brain are able to maintain their phenotypes, few changes in gene expression of components of signal transduction pathways were observed. MAP kinase and cGMP systems, which were altered in Alzheimer's disease, did not change in normally-aged brain. A change was also not observed in the gene encoding for an adapter protein to assemble signaling complexes. But a decrease by 27-fold of interferon-inducible protein 9-27 indicates some disturbance in intracellular signaling. This change can be understood in terms of a compensatory change to relay growth inhibitory signals that offset the potential risk for a dysregulated cell cycle.

Gene expression of coagulation factors in young MBEC changed in a direction opposite from that observed in Alzheimer's disease. In normally-aged MBEC, tissue factor was decreased by 3-fold, decay-accelerating factor was not changed, and complement factor H was increased by 4-fold. These changes may be associated with a local response to balance the procoagulation effects seen in the blood of normally-aged individuals.

Validation of the oligonucleotide array results was performed by a quantitative RT-PCR method for several genes including aminopeptidase N (CD13), aryl hydrocarbon nuclear receptor translocator-2 (ARNT2), CYP1B1, ephrin B2, and GAX. Western blot analysis of cell lysates (e.g., p16), ELISA of cell supernatants (e.g., secreted NGF), functional migration on extracellular matrix (e.g., integrins), coagulation assays (e.g., tissue factor activity, tissue plasminogen activator activity), and/or immunocytochemical analysis of brain in situ for several gene products including cyclin B1, aminopeptidase N, integrin β4, Notch-3, brain derived growth factor, etc. were performed. A general and consistent agreement was observed.

Proteomic studies (e.g., quantitative or semiquantitative Western blotting, ELISA and immunostaining) have confirmed that the changes in gene expression observed at the level of transcribed RNA are also detectable at the level of translated protein. In general, the direction of the change in gene expression (i.e., increased or decreased) is the same but the magnitude of any difference is not. This may reflect differences in the cell cultures or samples obtained therefrom, regulation at the level of protein translation or processing, saturation of the protein translation or processing machinery, or the like.

One or more master key genes (e.g., transcription factor, homeobox gene, or other regulatory gene) could be responsible for regulating gene expression in MBEC of patients with Alzheimer's disease. For example, VAG may provide a target to normalize the expression of one or more of the many genes in MBEC with altered expression.

For example, systemic or local delivery to the brain of one or more activators of VAG expression and/or one or more products encoded by VAG may represent an important new strategy to salvage or to protect neurons from neurodegeneration. Alternatively, compensating for the altered gene expression observed for any combination of VAG and other genes expressed in MBEC (e.g., neurotrophic factors and binding proteins for growth factors; detoxifying enzymes, and structural components of the vascular system that maintain the blood-brain barrier and active or passive transport therethrough; regulators of cell growth, entry into cell cycle, and cytokinesis) to substantially normalize the environment to that described for normal MBEC could benefit Alzheimer's disease patients and may exert neuroprotective effects. Pharmacogenomic and pharmacologic strategies to up regulate neurotrophic gene expression may be useful in treating Alzheimer's disease: for example, genetic manipulation of the microvasculature or delivery to the brain of a neurotrophic cocktail based on a mixture of gene products (e.g., secreted proteins) and/or recombinant material (e.g., antibodies, receptor ligands, agonists, and antagonists) for the genes that are down regulated or shutdown in MBEC of brains from Alzheimer's disease may represent a powerful strategy to prevent neuronal loss. Another alternative is inhibition or stimulation of enzyme activity to normalize the MBEC environment in Alzheimer's disease. Up regulation of these MBEC genes has the potential to improve dementia and neurodegeneration in Alzheimer's disease, and/or may arrest neurodegenerative disease by virtue of recouping the capability of MBEC to produce neurotrophic and other related factors necessary for health of neurons at an older age.

It is also envisioned that an inherited mutation, somatic mutation, or polygenic mutation of master key genes or VAG affecting MBEC, or generalized DNA damage, may cause Alzheimer's disease. Thus, one or more genetic mutations in the vascular system could be involved in pathogenesis of Alzheimer's disease. This represents another target for detection and correction of gene mutations to diagnose or treat Alzheimer's disease, respectively.

A viral gene transfer system based on VSV-pseudotyped MuLV-HSV has been developed for use in the microvasculature (Yu et al., Neurosurgery, 45-962-968, 1999). The identification of key regulatory genes and bioinformatics will lead to their incorporation into vectors suitable for gene transfer into MBEC. The candidate genes, for example, include master key genes and VAG as described above. Antisense strategies may restrain the activated cell cycle and control genes that are involved in chromosomal processing and segregation. The ability of gene therapy to revert the Alzheimer's disease phenotype of MBEC back to normal may be demonstrated in vitro using different cellular assays such as, for example, growth proliferation assay, release of ⁵¹Cr, TUNEL assay, FACS analysis of the cell cycle, migration capability assays on one or more substrates (e.g., MATRIGEL, laminin/collagen, vitronectin, fibronectin), capillary morphogenesis assays which assess the development of tube formation and organization of capillary networks (e.g., branching). Assays may be developed to determine the detoxifying capability of MBEC when exposed to “protoxicant” substrates, polyaromatic hydrocarbon compounds, or carcinogenic polutants following transfer of down regulated detoxification genes. Gene constructs may be designed to either produce an increase or decrease in a particular gene product or its metabolic/signaling pathway. Using either constitutive or drug-inducible expression vectors, master key genes or VAG may be expressed that will reverse or attenuate the pathogenic process in the microvessel endothelium and/or smooth muscle. Tissue-specific promoters can be configured in expression vectors to direct expression to the cell of interest. Repeated application of therapeutic genes is likely. Following in vitro studies, gene transfer may be performed in vivo directly to the vessel.

This discovery that multiple genes including growth factors, receptors, and cell cycle regulators are dysregulated in Alzheimer's disease presents opportunities to utilize FDA-approved drugs for inhibition of growth dysregulation. Antineoplastic drugs including, inter alia, alkylating agents (e.g., cytoxan), nucleoside analogs (e.g., FUdR), and antimetabolites (e.g., methotrexate) may be used to control abortive cell growth. Compounds with specific inhibitory activities against cyclins B and B1 may be used. Small molecules that block activated tyrosine kinase receptors may also be used. Molecules that reduce expression of VEGF-C, VEGF-related protein, tissue factor, and aryl hydrocarbon receptor nuclear translocator-2 or induce expression of VEGFR, angiopoetin-1 and tie-2 may also be used. Molecules that promote differentiated growth of MBEC can also be used such as those derived from proteins that are down regulated in MBEC from Alzheimer's disease patients including NGF, BDNF, FGF-5, BMP-1 FAP-α, IGFBP-5, IGFBP-1, IRH-α, or angiotensin-II receptor agonist. Proteins and derived smaller molecules from VAG genes may also be used. Vascular delivery and retention with endothelium of radiosensitizing agents followed by low-level external beam X-irradiation may also control the growth dysregulation.

High-throughput cell-based assays using fluorescent readouts of the reporter gene may be developed. Transcription factors that have been discovered to be abnormally regulated may be studied: e.g., C-MAF (AF055376) was decreased by 9-fold, FKHL7 (AF078096) was decreased by 6-fold, and DBY-alternative transcript 2 (AF00984) was decreased by 23-fold. These factors will either have known cis-acting elements through which they activate transcription or SAAB selection can be used to deduce them. One or more concatenated cis-acting elements may be ligated upstream of a fluorescent reporter gene, and then the construct can be transiently or stably transfected into mammalian cell lines of several types (e.g., those of endothelial or nonendothelial origin, derived from human and other species).

For example, first-order screening of compounds might identify those that either increase or decrease fluorescence. Second-order screening derives dose-dependent activities for each compound. Third-order screening of compounds in well-characterized cell models (e.g., MBEC from Alzheimer's disease patients) can be followed by in vivo testing in animal models of neurodegeneration.

It is envisioned using small therapeutic compounds that may either block MAPK and signals that are induced by phosphorylated MAPK or increase signaling within the GTP/cGMP pathway. Candidate compounds include PD98059, an inhibitor of MAPK, or molecules that act downstream in the signaling pathway such as NFκ-B inhibitors that are activated by MAPK (e.g., terolidinthiopyridine carbomaleate) or activated protein C (anticoagulant APC).

All references (e.g., articles, books, patents, and patent applications) cited above are indicative of the level of skill in the art and are incorporated by reference.

All modifications and substitutions that come within the meaning of the claims and the range of their legal equivalents are to be embraced within their scope. A claim using the transition “comprising” allows the inclusion of other elements to be within the scope of the claim; the invention is also described by such claims using the transitional phrase “consisting essentially of” (i.e., allowing the inclusion of other elements to be within the scope of the claim if they do not materially affect operation of the invention) and the transition “consisting” (i.e., allowing only the elements listed in the claim other than impurities or inconsequential activities which are ordinarily associated with the invention) instead of the “comprising” term. All three transitions can be used to claim the invention.

No particular relationship between or among limitations of a claim is meant unless such relationship is explicitly recited in the claim (e.g., the arrangement of components in a product claim or order of steps in a method claim is not a limitation of the claim unless explicitly stated to be so). Thus, all possible combinations and permutations of the individual elements disclosed herein are intended to be considered part of the invention.

From the foregoing, it would be apparent to a person of skill in this art that the invention can be embodied in other specific forms without departing from its spirit or essential characteristics. The described embodiments should be considered only as illustrative, not restrictive, because the scope of the legal protection provided for the invention will be indicated by the appended claims rather than by this specification.

What is claimed is:

1. A method of gene profiling in which at least a statistically significant change in gene expression is detected in endothelium comprising:
(a) providing RNA and/or protein from at least one cell derived from endothelium of a subject,
(b) measuring gene expression of at least five different genes in the at least one cells by quantitation of at least transcription and/or translation, and
(c) determining whether there is at least a statistically significant difference in gene expression of the at least five different genes in comparison to endothelium of one or more control subjects without Alzheimer's disease.

2. The method of claim 1, wherein the endothelium is from brain.

3. The method of claim 1, wherein the endothelium is from skin.

4. The method of claim 1, wherein the endothelium is from blood or vasculature.

5. The method of any one of claims 1-4 further comprising culturing at least one cell derived from the endothelium and obtaining RNA and/or protein from cultured cells to quantitate at least transcription and/or translation.

6. The method of claim 1, wherein at least transcription and/or translated is quantitated with an array.

7. The method of claim 1, wherein at least transcription and/or translated is quantitated with nucleic acid hybridization and/or antibody binding.

8. The method of claim 1, wherein transcription and translation are measured.

9. The method of claim 1, wherein the statistically significant difference in gene expression is determined with reference to a database containing gene profiling information of the one or more control subjects.

10. The method of any one of claims 1-9, wherein transcription and/or translation of at least ten different genes in the endothelium is measured.

11. The method of any one of claims 1-9, wherein transcription and/or translation of at least 100 different genes in the endothelium is measured.

12. The method of any one of claims 1-9, wherein transcription and/or translation of at least 1,000 different genes in the endothelium is measured.

13. The method of any one of claims 1-9, wherein there is at least a statistically significant difference in gene expression in at least five different genes.

14. The method of any one of claims 1-9, wherein there is at least a statistically significant difference in gene expression in at least ten different genes.

15. The method of any one of claims 1-9, wherein there is at least a statistically significant difference in gene expression in at least 25 different genes.

16. The method of any one of claims 1-9, wherein there is at least a statistically significant difference in gene expression in at least 50 different genes.

17. The method of any one of claims 1-9, wherein a 2.5-fold difference in transcription and/or translation is statistically significant.

18. The method of any one of claims 1-9, wherein a 5-fold difference in transcription and/or translation is statistically significant.

19. The method of any one of claims 1-9, wherein a 10-fold difference in transcription and/or translation is statistically significant.

20. The method of claim 1, wherein the subject is diagnosed to be at risk for or affected by Alzheimer's disease if there is at least a statistically significant difference in gene expression in at least five different genes which are increased or decreased in Alzheimer's disease.

21. The method of claim 1, wherein staging of disease or disease progression in the subject is determined by whether there is at least a statistically significant difference in gene expression for at least five different genes which are increased or decreased in Alzheimer's disease.

22. The method of claim 1, wherein treatment of the subject at least reduces statistically significant differences in gene expression for at least five different genes which are increased or decreased in Alzheimer's disease.

23. The method of claim 1, wherein candidate drugs are administered to the subject and are selected if there is at least a reduction in statistically significant differences in gene expression for at least five different genes which are increased or decreased in Alzheimer's disease.

24. The method of claim 1, wherein transcription and/or translation is measured for at least one gene selected from the group consisting of p16 inhibitor of G1 cyclin/cdk enzymes, AIM1, aminopeptidase N (CD13), ATP-binding cassette transporter 1 (ABCA1), aryl hydrocarbon receptor nuclear translocator 2 (ARNT2), brain derived neurotrophic factor (BDNF), collagen VIα, dihydrodiol dehydrogenase (DDH), dioxin-inducible cytochrome P450 (CYP1B1), DTK receptor tyrosine kinase, elastin, ephrin B2, glutamate transporters, growth arrest-specific 1 (GAS1), growth arrest-specific homeobox (GAX), integrins α7 and β4, low density lipoprotein receptor-related protein-1 (LRP-1), multidrug resistance protein-1 (MRP-1), N-methyltransferase (NNMT), nerve growth factor (NGF), Notch-3, and semaphorin III.

25. A database which is embodied on tangible medium comprising stored values for expression of at least five different genes from at least four positive controls with Alzheimer's disease and at least four negative controls without Alzheimer's disease.

26. A method of determining whether one or more cells manifest an Alzheimer's phenotype comprising:
(a) providing RNA and/or protein from the one or more cells;
(b) measuring transcription and/or translation of at least five different genes in the one or more cells, wherein the at least five different genes have been determined to have increased or decreased expression in subjects with Alzheimer's disease; and
(c) determining if the one or more cells manifest an Alzheimer's disease phenotype by whether there is at least a statistically significant difference in gene expression of the at least five different genes.

27. The method of claim 26, wherein the cell is not an endothelial cell.

28. The method of claim 26, wherein the cell is derived from an Alzheimer's disease subject if there is at least a statistically significant difference in gene expression of the at least five different genes.

29. The method of claim 26, wherein candidate drugs are administered to the one or more cells and selected if there is at least a reduction in statistically significant differences in gene expression for at least five different genes which are increased or decreased in subjects with Alzheimer's disease.

30. A drug selected by the method of claim 23 or 29.

31. A method of determining whether brain endothelium manifests an Alzheimer's phenotype by assaying apoptosis of cells derived from the brain endothelium.

31. A kit comprising an array, one or more positive controls with Alzheimer's disease, and one or more negative controls without Alzheimer's disease.
